SEQUENCE LISTING (103 sequences)

SEQ ID NO: 1 - DNA, 23 bp, Artificial Sequence
  mTmg for in vitro and in vivo editing
  misc_feature (21)..(23): PAM sequence
attatacgaagttatattaaggg

SEQ ID NO: 2 - DNA, 23 bp, Artificial Sequence
  Sftpc exon 1A
  misc_feature (21)..(23): PAM sequence
tcagagcccaggccccgataagg

SEQ ID NO: 3 - DNA, 23 bp, Artificial Sequence
  Sftpc exon 1B
  misc_feature (21)..(23): PAM Sequence
cagctgccttatcggggcctggg

SEQ ID NO: 4 - DNA, 23 bp, Artificial Sequence
  Sftpc exon 1C
  misc_feature (21)..(23): PAM sequence
ctcttgcagctgccttatcgggg

SEQ ID NO: 5 - DNA, 23 bp, Artificial Sequence
  Sftpc exon 5A
  misc_feature (21)..(23): PAM sequence
aggtgtctctcctacgggccagg

SEQ ID NO: 6 - DNA, 23 bp, Artificial Sequence
  Sftpc Exon 5B
  misc_feature (21)..(23): PAM sequence
ataggatccccctggcccgtagg

SEQ ID NO: 7 - DNA, 23 bp, Artificial Sequence
  Sftpc exon 5C
  misc_feature (21)..(23): PAM Sequence
ggtagaaaccgcagcgggacagg

SEQ ID NO: 8 - DNA, 23 bp, Artificial Sequence
  Sftpc exon 5D
  misc_feature (21)..(23): PAM Sequence
tacagacttccaccggtttctgg

SEQ ID NO: 9 - DNA, 23 bp, Artificial Sequence
  Sftpc exon 5E
  misc_feature (21)..(23): PAM Sequence
acaggaaagaccctccgcaaagg

SEQ ID NO: 10 - DNA, 20 bp, Artificial Sequence
  Sftpc gRNA exon 1A-C PCR forward
gcagtctgaccctaaggaac

SEQ ID NO: 11 - DNA, 20 bp, Artificial Sequence
  Sftpc gRNA exon 1A-C PCR reverse
ggactctccatcaggacctc

SEQ ID NO: 12 - DNA, 20 bp, Artificial Sequence
  Sftpc gRNA 5A-E PCR forward
ggaggaagggcatgatactg

SEQ ID NO: 13 - DNA, 20 bp, Artificial Sequence
  Sftpc gRNA 5A-E PCR reverse
ttgctctgttccccattacc

SEQ ID NO: 14 - DNA, 21 bp, Artificial Sequence
  mTmG Sanger Sequencing
cctgtccgttcgctttggaag

SEQ ID NO: 15 - DNA, 22 bp, Artificial Sequence
  mTmG PCR forward
aaatctgtgcggagccgaaatc

SEQ ID NO: 16 - DNA, 21 bp, Artificial Sequence
  mTmG PCR reverse
cctgtccgttcgctttggaag

SEQ ID NO: 17 - DNA, 20 bp, Artificial Sequence
  Sftpc Sanger Seqencing
ttgctctgttccccattacc

SEQ ID NO: 18 - DNA, 20 bp, Artificial Sequence
  Sftpc PCR forward
gcagtctgaccctaaggaac

SEQ ID NO: 19 - DNA, 20 bp, Artificial Sequence
  Sftpc PCR reverse
ttgctctgttccccattacc

SEQ ID NO: 20 - DNA, 19 bp, Artificial Sequence
  Sftpc unexcised PCR forward
acccaggtttgctcttgtt

SEQ ID NO: 21 - DNA, 22 bp, Artificial Sequence
  Sftpc unexcised PCR reverse
cttggctttgtagcttgtttgt

SEQ ID NO: 22 - DNA, 20 bp, Artificial Sequence
  Sftpc excised PCR forward
gagtttgcttacctcaccca

SEQ ID NO: 23 - DNA, 20 bp, Artificial Sequence
  Sftpc excised PCR reverse
ccaactctccaaaccctctc

SEQ ID NO: 24 - DNA, 24 bp, Artificial Sequence
  intronEhd2 forward primer
tctgtgtctaggactatcccaaat

SEQ ID NO: 25 - DNA, 20 bp, Artificial Sequence
  intronEhd2 reverse primer
gagggacgtctgtctcagaa

SEQ ID NO: 26 - DNA, 22 bp, Artificial Sequence
  intronLimk2 forward primer
tccacaccctttaggtcaatgc

SEQ ID NO: 27 - DNA, 20 bp, Artificial Sequence
  intronLimk2 reverse primer
tatgccagggatttgggcac

SEQ ID NO: 28 - DNA, 20 bp, Artificial Sequence
  intronActn4 forward primer
cctgtggagataagcagggc

SEQ ID NO: 29 - DNA, 20 bp, Artificial Sequence
  intronActn4 reverse primer
ctctcttgcctctcctccct

SEQ ID NO: 30 - DNA, 22 bp, Artificial Sequence
  intronCars forward primer
agacacctaaggaacaaggctg

SEQ ID NO: 31 - DNA, 21 bp, Artificial Sequence
  intronCars reverse primer
tccagtaaggacagctgggac

SEQ ID NO: 32 - DNA, 20 bp, Artificial Sequence
  intergenicWnt9a-Prss38 forward primer
cctctgcacagaaggtgctt

SEQ ID NO: 33 - DNA, 20 bp, Artificial Sequence
  intergenicWnt9a-Prss38 reverse primer
taagcttccagctggcttcc

SEQ ID NO: 34 - DNA, 22 bp, Artificial Sequence
  intronHs6st3 forward primer
gtcccacagacattgattctca

SEQ ID NO: 35 - DNA, 21 bp, Artificial Sequence
  intronHs6st3 reverse primer
aatctagatctgcctggaccc

SEQ ID NO: 36 - DNA, 21 bp, Artificial Sequence
  inrtonPrkab1 forward primer
acaggagactcactacacggt

SEQ ID NO: 37 - DNA, 20 bp, Artificial Sequence
  intronPrkab1 reverse primer
aaatagggggcagggaccat

SEQ ID NO: 38 - DNA, 21 bp, Artificial Sequence
  exonGpc2 forward primer
ggagatcatgtcagacacccc

SEQ ID NO: 39 - DNA, 20 bp, Artificial Sequence
  exonGpc2 reverse primer
tggaagaaatgtggtcagcg

SEQ ID NO: 40 - DNA, 21 bp, Artificial Sequence
  intergenicGpa33-Mael forward primer
tttcatgctccttgttgtcgg

SEQ ID NO: 41 - DNA, 20 bp, Artificial Sequence
  intergenicGpa33-Mael reverse Primer
ttgaatccgggctctatggt

SEQ ID NO: 42 - DNA, 20 bp, Artificial Sequence
  intronWwox forward primer
cctccgctgaggtctgaagt

SEQ ID NO: 43 - DNA, 21 bp, Artificial Sequence
  intronWwox reverse primer
ggcccaactgaaccctaagat

SEQ ID NO: 44 - DNA, 20 bp, Artificial Sequence
  intergenicCt573086.1-Hlcs forward primer
gctccctgcagaaggatcac

SEQ ID NO: 45 - DNA, 20 bp, Artificial Sequence
  intergenicCt573086.1-Hlcs reverse primer
gacatccacatggcctgttc

SEQ ID NO: 46 - DNA, 20 bp, Artificial Sequence
  intergenicU6atac-7SK forward primer
tcaaggtggagaaggcatgg

SEQ ID NO: 47 - DNA, 25 bp, Artificial Sequence
  intergenicU6atac-7SK reverse primer
tcctaaaccagtatgaaaagcttcc

SEQ ID NO: 48 - DNA, 20 bp, Artificial Sequence
  intergenicGm17566-AC124613.1 forward primer
ggaagcggattgctgacatc

SEQ ID NO: 49 - DNA, 21 bp, Artificial Sequence
  intergenicGm17566-AC124613.1 reverse primer
caggggaggaactagagggaa

SEQ ID NO: 50 - DNA, 21 bp, Artificial Sequence
  intronHs6st3 forward primer
tgaggctcataggttcacgtc

SEQ ID NO: 51 - DNA, 20 bp, Artificial Sequence
  intronHs6st3 reverse primer
acagagactcgaatccccca

SEQ ID NO: 52 - DNA, 20 bp, Artificial Sequence
  intergenicIrf2bp2-Tomm20 forward primer
gccacacagaagggggttag

SEQ ID NO: 53 - DNA, 20 bp, Artificial Sequence
  intergenicIrf2bp2-Tomm20 reverse primer
ttaggcctgcatgggaaagg

SEQ ID NO: 54 - DNA, 20 bp, Artificial Sequence
  intronSirpa forward primer
ttcctgctgaatgccgtcac

SEQ ID NO: 55 - DNA, 23 bp, Artificial Sequence
  intronSirpa reverse primer
tgtgatgctttagggaaagatgc

SEQ ID NO: 56 - DNA, 21 bp, Artificial Sequence
  intergenicRmst-U7 forward primer
tcagatgtgcaggtccagaga

SEQ ID NO: 57 - DNA, 20 bp, Artificial Sequence
  intergenicRmst-U7 reverse primer
atccttgtgcttgcccctat

SEQ ID NO: 58 - DNA, 21 bp, Artificial Sequence
  intronBmp6 forward primer
cacactgctcctctcctgatt

SEQ ID NO: 59 - DNA, 21 bp, Artificial Sequence
  intronBmp6 reverse primer
acacagcatggagttcaagca

SEQ ID NO: 60 - DNA, 20 bp, Artificial Sequence
  intronKcnj10 forward primer
cagccacttcaccttcgagc

SEQ ID NO: 61 - DNA, 23 bp, Artificial Sequence
  intronKcnj10 reverse primer
gatggaagacccgaggtgaataa

SEQ ID NO: 62 - DNA, 20 bp, Artificial Sequence
  intronFras1 forward primer
ggctatctttggctcgtcca

SEQ ID NO: 63 - DNA, 21 bp, Artificial Sequence
  intronFras1 reverse primer
tcaagagggttccagtggatt

SEQ ID NO: 64 - DNA, 23 bp, Artificial Sequence
  intronEhd2 sgRNA Sftpc target site
  misc_feature (21)..(23): PAM Sequence
ttggagcccaggccccaataggg

SEQ ID NO: 65 - DNA, 23 bp, Artificial Sequence
  intronLimk2 sgRNA Sftpc target site
  misc_feature (21)..(23): PAM Sequence
tcaaaccccaggcccagattagg

SEQ ID NO: 66 - DNA, 23 bp, Artificial Sequence
  intronActn4 sgRNA Sftpc target site
  misc_feature (21)..(23): PAM Sequence
gcagaggccaggcccagattagg

SEQ ID NO: 67 - DNA, 23 bp, Artificial Sequence
  intronCars sgRNA Sftpc target site
  misc_feature (21)..(23): PAM Sequence
tctgatcccaggcccagttaagg

SEQ ID NO: 68 - DNA, 23 bp, Artificial Sequence
  intergenicWnt9a-Prss38 sgRNA Sftpc target site
  misc_feature (21)..(23): PAM Sequence
acagagcacaggcccagaaaggg

SEQ ID NO: 69 - DNA, 23 bp, Artificial Sequence
  intronHs6st3 sgRNA Sftpc target site
  misc_feature (21)..(23): PAM Sequence
taagagccaaggccccgacaggg

SEQ ID NO: 70 - DNA, 23 bp, Artificial Sequence
  intronPrkab1 sgRNA Sftpc target site
  misc_feature (21)..(23): PAM Sequence
tcagagcacaggctcagaaacgg

SEQ ID NO: 71 - DNA, 23 bp, Artificial Sequence
  exonGpc2 sgRNA Sftpc target site
  misc_feature (21)..(23): PAM Sequence
ccagagcccaggaacagataagg

SEQ ID NO: 72 - DNA, 23 bp, Artificial Sequence
  intergenicGpa33-Mael sgRNA Sftpc target site
  misc_feature (21)..(23): PAM Sequence
tcagagccaaggtcccactatgg

SEQ ID NO: 73 - DNA, 23 bp, Artificial Sequence
  intronWwox sgRNA Sftpc target site
  misc_feature (21)..(23): PAM Sequence
tcaaagcccaggcccagcttggg

SEQ ID NO: 74 - DNA, 23 bp, Artificial Sequence
  intergenic CT573086.1-Hlcs sgRNA Sftpc target site
  misc_feature (21)..(23): PAM Sequence
acaggacccacctggcctgttgg

SEQ ID NO: 75 - DNA, 23 bp, Artificial Sequence
  intergenic U6atac-7SK sgRNA Sftpc target site
  misc_feature (21)..(23): PAM Sequence
ataggatactcatggcccattgg

SEQ ID NO: 76 - DNA, 23 bp, Artificial Sequence
  intergenicGm17566-AC124613.1 sgRNA Sftpc target site
  misc_feature (21)..(23): PAM Sequence
ttaagatctgcctggcccgtggg

SEQ ID NO: 77 - DNA, 23 bp, Artificial Sequence
  intronHs6st3 sgRNA Sftpc target site
  misc_feature (21)..(23): PAM Sequence
ataggaggccccaggaccgtagg

SEQ ID NO: 78 - DNA, 23 bp, Artificial Sequence
  intergenicIrf2bp2-Tomm20 sgRNA Sftpc target site
  misc_feature (21)..(23): PAM Sequence
ataggactgccctggccctttgg

SEQ ID NO: 79 - DNA, 23 bp, Artificial Sequence
  intronSirpasgRNA Sftpc target site
  misc_feature (21)..(23): PAM Sequence
atgggatccccatggaccgaggg

SEQ ID NO: 80 - DNA, 23 bp, Artificial Sequence
  intergenicRmst-U7 sgRNA Sftpc target site
  misc_feature (21)..(23): PAM Sequence
attggattccccaggcccgaagg

SEQ ID NO: 81 - DNA, 23 bp, Artificial Sequence
  intronBmp6 sgRNA Sftpc target site
  misc_feature (21)..(23): PAM Sequence
atagaaacccccaggcccctcgg

SEQ ID NO: 82 - DNA, 23 bp, Artificial Sequence
  intronKcnj10 sgRNA Sftpc target site
  misc_feature (21)..(23): PAM Sequence
atcggatccccctaaccctttgg

SEQ ID NO: 83 - DNA, 23 bp, Artificial Sequence
  intronFras1 sgRNA SFtpc target site
  misc_feature (21)..(23): PAM Sequence
atggactccccctggcccttagg

SEQ ID NO: 84 - DNA, 23 bp, Artificial Sequence
  I73T-SFTPC-gRNA1
  misc_feature (21)..(23): PAM Sequence
gtgctcatctccagaacctgggg

SEQ ID NO: 85 - DNA, 23 bp, Artificial Sequence
  I73T-SFTPC-gRNA2
  misc_feature (21)..(23): PAM Sequence
agtgctcatctccagaacctggg

SEQ ID NO: 86 - DNA, 23 bp, Artificial Sequence
  I73T-SFTPC-gRNA3
  misc_feature (21)..(23): PAM Sequence
cagtgctcatctccagaacctgg

SEQ ID NO: 87 - DNA, 23 bp, Artificial Sequence
  I73T-SFTPC-gRNA4
  misc_feature (21)..(23): PAM Sequence
caggttctggagatgagcactgg

SEQ ID NO: 88 - DNA, 23 bp, Artificial Sequence
  I73T-SFTPC-gRNA5
  misc_feature (21)..(23): PAM Sequence
aggttctggagatgagcactggg

SEQ ID NO: 89 - DNA, 23 bp, Artificial Sequence
  I73T-SFTPC-gRNA6
  misc_feature (21)..(23): PAM Sequence
ggttctggagatgagcactgggg

SEQ ID NO: 90 - DNA, 23 bp, Artificial Sequence
  I73T-SFTPC-gRNA7
  misc_feature (21)..(23): PAM Sequence
ggagatgagcactggggcgccgg

SEQ ID NO: 91 - DNA, 22 bp, Artificial Sequence
  I73T-SFTPC-gRNA8
  misc_feature (21)..(22): PAM Sequence
gagatgagcactggggcgccgg

SEQ ID NO: 92 - DNA, 22 bp, Artificial Sequence
  I73T-SFTPC-gRNA9
  misc_feature (21)..(22): PAM Sequence
agatgagcactggggcgccgga

SEQ ID NO: 93 - DNA, 23 bp, Artificial Sequence
  I73T-SFTPC-gRNA10
  misc_feature (21)..(23): PAM Sequence
gagatgagcactggggcgccgga

SEQ ID NO: 94 - DNA, 22 bp, Artificial Sequence
  I73T-SFTPC-gRNA11
  misc_feature (21)..(22): PAM Sequence
atgagcactggggcgccggaag

SEQ ID NO: 95 - DNA, 22 bp, Artificial Sequence
  I73T-SFTPC-gRNA12
  misc_feature (21)..(22): PAM Sequence
cactggggcgccggaagcccag

SEQ ID NO: 96 - DNA, 22 bp, Artificial Sequence
  I73T-SFTPC-gRNA13
  misc_feature (21)..(22): PAM Sequence
tctggagatgagcactggggcg

SEQ ID NO: 97 - DNA, 22 bp, Artificial Sequence
  I73T-SFTPC-gRNA14
  misc_feature (21)..(22): PAM Sequence
gttctggagatgagcactgggg

SEQ ID NO: 98 - DNA, 22 bp, Artificial Sequence
  I73T-SFTPC-gRNA15
  misc_feature (21)..(22): PAM Sequence
caggttctggagatgagcactg

SEQ ID NO: 99 - DNA, 23 bp, Artificial Sequence
  CFTR-Del508-gRNA1
  misc_feature (21)..(23): PAM Sequence
accattaaagaaaatatcattgg

SEQ ID NO: 100 - DNA, 23 bp, Artificial Sequence
  CFTR-Del508-gRNA2
  misc_feature (21)..(23): PAM Sequence
accaatgatattttctttaatgg

SEQ ID NO: 101 - DNA, 23 bp, Artificial Sequence
  CFTR-Del508-gRNA3
  misc_feature (21)..(23): PAM Sequence
tctgtatctatattcatcatagg

SEQ ID NO: 102 - DNA, 23 bp, Artificial Sequence
  CFTR-Del508-gRNA4
  misc_feature (21)..(23): PAM Sequence
aatggtgccaggcataatccagg

SEQ ID NO: 103 - DNA, 23 bp, Artificial Sequence
  CFTR-Del508-gRNA5
  misc_feature (21)..(23): PAM Sequence
agtttcttacctcttctagttgg

(19) United States
(12) Patent Application Publication
(10) Pub. No.: US 2022/0275402 A1
(43) Pub. Date: Sep. 1, 2022

(54) COMPOSITIONS AND METHODS FOR IN UTERO GENE EDITING FOR MONOGENIC LUNG DISEASE

(71) Applicants: THE CHILDREN'S HOSPITAL OF PHILADELPHIA, Philadelphia, PA (US); THE TRUSTEES OF THE UNIVERSITY OF PENNSYLVANIA, Philadelphia, PA (US)

(72) Inventors: William H. Peranteau, Philadelphia, PA (US); Edward E. Morrisey, Newtown Square, PA (US); Michael F. Beers, Philadelphia, PA (US); Kiran Musunuru, Philadelphia, PA (US)

(73) Assignees: THE CHILDREN'S HOSPITAL OF PHILADELPHIA, Philadelphia, PA (US); THE TRUSTEES OF THE UNIVERSITY OF PENNSYLVANIA, Philadelphia, PA (US)

(21) Appl. No.: 17/601,581

(22) PCT Filed: Apr. 6, 2020
(86) PCT No.: PCT/US2020/026949

§ 371 Date: Oct. 5, 2021

Related U.S. Application Data
(60) Provisional application No. 62/830,032, filed on Apr. 5, 2019.

(51) Int. Cl. C12N 15/90; C07K 14/705; C07K 14/81; C12N 9/22; A61K 9/00

(52) U.S. Cl. CPC C12N 15/907; C07K 14/705; C07K 14/8121; C12N 9/22; A61K 9/0019; A61K 9/007

(57) ABSTRACT

Compositions and methods for in utero gene editing in mammalian lung cells are disclosed.

### CROSS REFERENCE TO RELATED APPLICATION

This application is a § 371 of International Application No, PCT/US2020/026949, filed Apr. 6, 2020, which claims priority to U.S. Provisional Application No. 62/830,032 filed on Apr. 5, 2019, the entire contents of each being incorporated herein by reference.

### GRANT SUPPORT STATEMENT

This invention was made with government support under Grant Numbers 1U01HL134745, UL1-TR001878 and HL119436 awarded by the National Institutes of Health and Grant Number 1I01BX001176 awarded by the US Dept. of Veteran Affairs. The US government has certain rights in the invention.

### INCORPORATION-BY-REFERENCE OF MATERIAL SUBMITTED IN ELECTRONIC FORM

Incorporated herein by reference in its entirety is the Sequence Listing submitted via EFS-Web as a text file named PeranteauSequenceListing.txt, created Oct. 5, 2021 and having a size of 24,575 bytes.

### FIELD OF THE INVENTION

This invention relates to the fields of genetic disease and gene editing technology. More specifically, the invention provides compositions and methods for correcting gene sequences in utero, thereby curing or ameliorating symptoms of genetic lung disease before or after birth.

### BACKGROUND OF THE INVENTION

Several publications and patent documents are cited throughout the specification in order to describe the state of the art to which this invention pertains. Each of these citations is incorporated by reference herein as though set forth in full.

Congenital genetic lung diseases, such as inherited surfactant protein (SP) syndromes, cystic fibrosis and alpha-1 antitrypsin deficiency, are a source of morbidity and mortality for which no definitive treatment options exist (1-4). These disorders present with a spectrum of severity and timing of onset. Some, such as cystic fibrosis and alpha-1 antitrypsin deficiency, present in late childhood or early adulthood with subsequent disease progression and shortened life expectancy (5, 6). Alternatively, mutations in SP genes can cause respiratory failure at birth and perinatal death or chronic diffuse lung disease. Genetic mutations in one of three surfactant system genes, SFTPB, SFTPC, or ATP-binding cassette protein member 3 (ABCA3), result in either a loss of function phenotype through disruption of surfactant metabolism or its biophysical activity (SP deficiency syndrome) or a toxic gain of function phenotype from disrupted lung development or diffuse parenchymal lung disease perpetrated by cytosolic accumulation of abnormal SP conformers in alveolar type 2 (AT2) cells. Heterozygous mutations in SFTPC are a primary cause of children's interstitial lung disease (chILD). The age of onset and severity of disease due to SFTPC mutations depends on the specific mutation and varies from severe respiratory failure in neonates to idiopathic pulmonary fibrosis in adulthood (7, 8). Unlike surfactant deficiency of prematurity, the inherited forms of SP disease do not respond to exogenous surfactant, anti-inflammatory, or anti-fibrotic therapies. Treatment options for patients presenting with neonatal respiratory failure are limited to palliative care or pediatric lung transplant which is limited by organ availability (9, 10). Thus, there is an urgent need for novel therapies for early correction of lethal genetic lung disorders including SP syndromes.

### SUMMARY OF THE INVENTION

In accordance with the present invention, compositions and a CRISPR-Cas system-mediated genome editing method are disclosed. An exemplary method comprises introducing into a eukaryotic cell containing and expressing a DNA molecule having a target sequence and encoding at least one mutated gene product in the lung, an engineered, non-naturally occurring CRISPR-Cas system comprising one or more vectors comprising a) a first regulatory element operable in a eukaryotic cell operably linked to at least one nucleotide sequence encoding a CRISPR-Cas system guide RNA that hybridizes with the target sequence, and b) a second regulatory element operable in a eukaryotic cell operably linked to a nucleotide sequence encoding a Cas9 protein or variant thereof, wherein components (a) and (b) are located on same or different vectors of the system or are affixed molecules which effect nucleic acid delivery into mammalian cells, whereby expression of the at least one gene product is altered through the CRISPR-Cas system acting via the DNA molecule comprising the guide RNA directing sequence-specific binding of the CRISPR-Cas system, causing genome editing to remove one or more undesired mutations; and, wherein the Cas9 protein and the guide RNA do not naturally occur together, and said guide strand targets a gene in fetal or post-natal lung selected from the group consisting mutated SFTPB, SFTPC, ABCA3, SERPINA1, and CFTR. In certain embodiments of the method, the CRISPR-Cas system further comprises one or more nuclear localization sequence(s) and, or a tracr sequence.

In some embodiments that the Cas9 protein is codon optimized for expression in a human lung cell.

The invention also provides a method of treating a monogenic lung disease in a subject in need thereof comprising editing a gene in a lung cell of the subject using the CRISPR-Cas system described above. The subject may be selected from a fetal, post-natal, pediatric or adult subject.

Also disclosed is a CRISPR/Cas nuclease comprising a single guide RNA that binds to a target site in a mutated gene causing monogenic lung disease, wherein the nuclease cleaves and inactivates the mutated gene. In one aspect, the gene is SFTPC. Mammalian cells comprising the nuclease are also within the scope of the invention.

In another embodiment, a method of inactivating an endogenous gene causing monogenic disease in a lung cell is provided. An exemplary method comprises the steps of: administering to the cell a CRISPR/Cas nuclease described above, wherein the nuclease cleaves and inactivates a gene causing lethal monogenic lung disease. In preferred embodiments the CRISPR-Cas system is provided such that is it present transiently in the subject.

Also disclosed are guide strands useful for targeting surfactant protein C and the CFTR gene. Kits for practicing the methods disclosed are also within the scope of the invention.

### BRIEF DESCRIPTION OF THE DRAWINGS

FIGS. 1A-1G. Intra-amniotic delivery of CRISPR-Cas9 results in pulmonary gene editing. (FIG. 1A) Schematic representation of intra-amniotic route of fetal lung gene editing. (FIG. 1B) Experimental design of gene editing in R26^{mTmG/+} mice. (FIG. 1C) Fluorescent stereomicroscopy, using a filter to detect tdTomato and EGFP, of lungs from R26^{mTmG/+} mice injected with Ad.Cre, Ad.mTmG, or Ad.Null. (FIG. 1D) Immunohistochemistry for EGFP and tdTomato expression in the proximal airway and distal air saccules of lungs from R26^{mTmG/+} mice injected with Ad.Cre, Ad.mTmG, or Ad.Null. White arrowheads indicate EGFP staining. (FIG. 1E) PCR assay using primers to detect the on-target editing in DNA isolated from E19 lungs of R26^{mTmG/+} mice injected with Ad.Cre, Ad.mTmG, or Ad.Null. Edited band=545 bp; unedited band=2951 bp; n=2-6 per group. One fetus that was injected with Ad.mTmG and lacked notable EGFP fluorescence (GFP-) was also negative for gene editing by PCR, indicating a likely technical failure at the time of injection. (FIG. 1F) Sanger sequencing of the 545 bp edited mTmG PCR product from an R26^{mTmG/+} mouse injected with Ad.mTmG. (FIG. 1G) Sanger sequencing of the 545 bp cre-recombined mTmG PCR product from an R26^{mTmG/+} mouse injected with Ad.Cre. Scale bars=1000 μm for C and 50 μm for D. IA, intra-amniotic; E, gestational day.

FIGS. 2A-2D. R26^{mTmG} gene locus of interest and lack of gene editing in non-pulmonary organs after IA delivery. (FIG. 2A) Schematic of genomic sequence of mTmG locus of interest. mTmG sgRNA sequence (blue) and PAM site targeting the loxP sites (purple) that flank the tdTomato (red) and poly A stop cassette. EGFP sequence (green) 3′ to the sgRNA targeting loxP site 2 is expressed after gene editing and NHEJ. (FIG. 2B) PCR analysis for the 545 bp edited mTmG band in DNA isolated at 1 month of age from the indicated organs of E16 IA injected Ad.mTmG recipients. +C=Ad.Cre injected R26^{mTmG} fetus; −C=Ad.Null injected R26^{mTmG} fetus; Arrow indicates the 545 bp detected in DNA from the stomach. (FIG. 2C) Representative images of nonpulmonary organs from prenatally injected Ad.mTmG R26_{mTmG} fetuses assessed by IHC for EGFP at P30. White arrowhead indicates EGFP⁺ cells in the stomach. (FIG. 2D) Tile image of an E19 fetus injected with an Ad vector containing the EGFP transgene at E16 via the IA route, depicting EGFP⁺ cells in the nasopharynx (yellow arrowhead) and lung (white arrowhead). Scale bars=50 μm for C and 1500 μm for D. PAM, protospacer adjacent motif.

FIGS. 3A-3E. Intra-amniotic delivery of CRISPR-Cas9 targets pulmonary epithelial cells for gene editing. (FIG. 3A) FACS plots of lungs harvested at E19 after IA injection of Ad.mTmG, Ad.Cre, or Ad.Null at E16. Each row shows representative FACS plots from a single lung. (FIG. 3B) Quantitation of cell type-specific gene editing using FACS analysis for EGFP cells within each major pulmonary cell type after IA injection of Ad.mTmG and Ad.Cre; n=5 per group. (FIG. 3C) EGFP⁺ gene-edited and Cre-recombined cells depicted by white arrowheads within subsets of pulmonary epithelial cells marked by AQP5, SFTPC, SCGB1A1, and FOXJ1. (FIG. 3D) Quantification of gene-edited airway and alveolar epithelial cells after Ad.mTmG IA delivery. (FIG. 3E) Quantification of Cre-recombined airway and alveolar epithelial cells after Ad.Cre IA delivery. n=2-5 per group. Epi, epithelial; Endo, endothelial; Mes, mesenchymal; IA, intra-amniotic; AT1, alveolar type 1; AT2, alveolar type 2. Scale bars=50 μm.

FIGS. 4A-4B. Distribution of gene-edited cells in the lung. E16 R26^{mTmG} fetuses were injected IA with Ad.mTmG and lungs were harvested at E19 for analyses by IHC and flow cytometry for EGFP expression indicative of editing. (FIG. 4A) Representative tile image of an E19 lung section with focused evaluation of the airway, saccules, and blood vessels. White arrowheads indicate representative EGFP⁺ cells. (FIG. 4B) Gates used for flow cytometric analysis in R26^{mTmG} mouse model and an isotype negative control. IHC, immunohistochemistry; AW, airway; Sac, saccule; BV, blood vessel. Scale bars=50 μm.

FIGS. 5A-5D. Pulmonary epithelial cell gene editing is stable over time. (FIG. 5A) Experimental design for longer term analysis of pulmonary epithelial cell gene editing after IA Ad.mTmG delivery at E16. (FIG. 5B) Quantification of edited pulmonary epithelial, endothelial, and mesenchymal cell types at E19, P7, P30, and 6 months by FACS analysis. (FIG. 5C) Quantification of gene editing in individual pulmonary cell types at E19, P7, P30, and 6 months by IHC. (FIG. 5D) Schematic summary of fetal pulmonary cells that underwent gene editing after intra-amniotic delivery of CRISPR-Cas9 targeting the mT gene. n=3-5 per group; ** p<0.01, and * p<0.05 by one-way ANOVA followed by Tukey's multiple comparison test. IA, intra-amniotic; IHC, immunohistochemistry; AT1, alveolar type 1; AT2, alveolar type 2.

FIGS. 6A-6D. Gene editing in pulmonary cell types. Genomic DNA from sorted pulmonary EPCAM⁺ (epithelial) cells, CD31⁺ (endothelial) cells, and EPCAM⁻CD31⁻ (mesenchymal) cells was evaluated by PCR for the presence of the 545 bp edited mTmG band indicative of editing and NHEJ following injection of E16 R26^{mTmG} fetuses with (FIG. 6A) Ad.mTmG, (FIG. 6B) Ad.Cre, or (FIG. 6C) Ad.Null. (FIG. 6D) FACS plots and quantification of tdTomato and EGFP double negative cells. n=3 per group; NS=not significant: p=0.2 by unpaired two-tailed Student's t-test. +C, positive control=lung DNA from a recipient of Ad.Cre; −C, negative control=lung DNA from a recipient of Ad.Null.

FIGS. 7A-7M. Prenatal gene editing in Sftpc^{I73T} mice decreases mutant SP-C^{I73T} pro-protein and improves lung alveolarization. (FIG. 7A) Schematic representation of Sftpc^{I73T} mutation causing intracellular accumulation SP-C^{I73T} pro-protein resulting in AT2 cell injury and potential cell rescue with CRISPR-Cas9-mediated excision of Sftpc^{I73T}. (FIG. 7B) Fluorescent stereomicroscopy, using a filter to detect EGFP, of an E19 fetus (outlined by white dashed line) after IA injection of Ad.Sftpc.GFP at E16 shows green fluorescence in the chest region. (FIG. 7C) Fluorescent stereomicroscopy, using a filter to detect EGFP, of lungs at E19 after E16 IA injection of Ad.Sftpc.GFP. (FIG. 7D) IHC for EGFP of lung parenchyma at E19 after E16 IA injection of Ad.Sftpc.GFP. (FIG. 7E) FACS analysis to assess EGFP expression in all pulmonary cells and pulmonary epithelial cells (EPCAM⁺ cells) from E19 fetuses after E16 IA injection of Ad.Sftpc.GFP. n=10-11 per group. (FIG. 7F) PCR analysis of DNA from E19 lung epithelial cells (EPCAM⁺ sorted cells) of E16 Ad.Sftpc.GFP IA injected fetuses. Edited Sftpc band=605 bp. −C and +C=negative and positive controls consisting of nontransfected mouse neuro-2a cells and mouse neuro-2a cells co-transfected with plasmids containing spyCas9, sgRNA1-A and sgRNA5-B respectively. (FIG. 7G) Schematic of Sftpc^{I73T} experimental design. (FIG. 7H) Excision of the mutant Sftpc allele in AT2 cells was assessed by IHC. Lungs of E19 Sftpc^{I73T/WT} mice were assessed for expression of SFTPB and HA after E16 IA injection of Ad.Null.GFP or Ad.Sftpc.GFP. SFTPB⁺HA⁻ (yellow arrowheads indicate representative cells)=excision; SFTPB⁺HA⁺ (white arrowheads indicate representative cells)=no excision; Control=uninjected WT E19 lungs. (FIG. 7I) The percentage of SFTPB⁺HA⁻ cells on IHC was quantified. (FIG. 7J) Lung IHC for HOPX at E19 to assess AT1 cell morphology and spreading in Sftpc^{I73T/WT} mice injected with Ad.Null.GFP or Ad.Sftpc.GFP at E16. (FIG. 7K) The internuclear distance was measured to quantify AT1 spreading. (FIG. 7L) H and E staining of lungs from E19 Sftpc^{I73T/WT} mice injected at E16 with Ad.Null.GFP or Ad.Sftpc.GFP to assess alveolarization/sacculation. (FIG. 7M) The mean linear intercept was calculated to assess alveolarization. n=3-4 per group; ## p<0.0001, ** p<0.01, and * p<0.05 by one-way ANOVA followed by Tukey's multiple comparison test; IHC, immunohistochemistry; WT, wild-type; IA, intra-amniotic; AT1, alveolar type 1; AT2, alveolar type 2. Scale bars=50 μm.

FIGS. 8A-8K. Selection of sgRNAs for excision of Sftpc gene and in vivo gene editing in C57BL/6 and Sftpc^{I73T/WT} mice. (FIG. 8A) Schematic of genomic sequence of Sftpc^{WT} locus of interest. sgRNA sequence (blue) and PAM site targeting 5′ to exon 1 and 3′ to exon 5 to excise the Sftpc gene. (FIG. 8B) sgRNAs were screened in mouse neuro-2a cells and editing assessed by Surveyor assay. (FIG. 8C) Schematic of sgRNAs used to excise the Sftpc gene. (FIG. 8D) Mouse neuro-2a cells were co-transfected with plasmids containing sgRNA 1-A and 5-B and editing assessed by PCR. Edited band=605 bp. (FIG. 8E) E16 C57BL/6 fetuses were injected IA with Ad.Sftpc.GFP and lungs were assessed at E19 by fluorescent stereomicroscope for EGFP expression and DNA isolated for PCR analysis using primers to amplify the Sftpc edited band (edited band=605 bp). (FIG. 8F) Sanger sequencing demonstrates editing and NHEJ three nucleotides 5′ to PAM sequence. (FIG. 8G) Schematic of experimental design for analysis of gene editing after IA injection at earlier and later gestation periods. (FIG. 8H) Percentage of EGFP⁺ pulmonary cells was assessed by FACS analysis at E19 after IA injection of Ad.Sftpc.GFP at different gestational ages. n=3-12 per group; # p<0.001 and ## p<0.0001, NS=not significant: p=0.99 by one-way ANOVA followed by Tukey's multiple comparison test. (FIG. 8I) Quantitative real-time PCR showing the rate of gene editing in whole lung DNA at E14, E16, and E17. (FIG. 8J) Schematic of Sftpc^{I73T} adult mice and progeny after crossing with FlpO⁺⁺ mice and site of gene editing. (FIG. 8K) PCR analysis for the Sftpc edited band of DNA from E19 lungs after IA injection of E16 Sftpc^{I73T/WT} mice with Ad.Sftpc.GFP or Ad.Null.GFP. Edited band=605 bp; unedited band=3950 bp. −C and +C=negative and positive controls consisting of nontransfected mouse neuro-2a cells and mouse neuro-2a cells co-transfected with plasmids containing spyCas9, sgRNA1-A and sgRNA5-B respectively; IA, intra-amniotic; C, control; WT, wild-type.

FIGS. 9A-9I. Prenatal gene editing in Sftpc^{I73T} mutant mice improves survival. (FIG. 9A) Schematic of experimental design for survival analysis of Sftpc^{I73T} mutant mice. (FIG. 9B) Survival of C57BL/6 mice injected at E16 with Ad.Sftpc.GFP (blue), gene-edited Sftpc^{I73T/WT} mice injected with Ad.Sftpc.GFP at E16 (red), Sftpc^{I73T/WT} mice injected with Ad.Null.GFP at E16 (green), and un-injected Sftpc^{I73T/WT} mice (purple). (FIG. 9C) The survival frequency of Ad.Sftpc.GFP treated Sftpc^{I73T/WT} mice was normalized to the survival rate of control C57BL/6 treated mice at 1 week of age. n=20-87 per group; **p<0.01 by log-rank (Mantel-Cox) test for comparison of survival curves. (FIG. 9D) H and E staining of lungs from 1-week-old Sftpc^{I73T/WT} mice and C57BL/6 mice injected with Ad.Sftpc.GFP at E16 and uninjected WT C57BL/6 mice was performed to assess alveolarization. (FIG. 9E) The mean linear intercept was calculated to assess alveolarization. (FIG. 9F) IHC for SFTPB and HA was performed on lungs from 1-week-old Sftpc^{I73T/WT} mice and C57BL/6 mice injected with Ad.Sftpc.GFP at E16 and uninjected WT C57BL/6 mice to assess AT2 cell morphology and excision of the mutant Sftpc allele in AT2 cells. SFTPB⁺HA⁻ (yellow arrowheads indicate representative cells)=excision; SFTPB⁺HA⁺ (white arrowheads indicate representative cells)=no excision. (FIG. 9G) The percentage of SFTPB⁺HA⁻ cells, indicative of gene-edited cells in Ad.Sftpc.GFP injected Sftpc^{I73T/WT} mice, was quantified on IHC. (FIG. 9H) IHC for HOPX was performed to assess AT1 cell morphology in 1-week-old Sftpc^{I73T/WT} mice and C57BL/6 mice injected with Ad.Sftpc.GFP at E16 and uninjected WT C57BL/6 mice. (FIG. 9I) The internuclear distance was calculated to assess AT1 cell spreading. n=3-4 per group; ^{##}p<0.0001 by one-way ANOVA followed by Tukey's multiple comparison test. WT, wild type; IHC, immunohistochemistry; AT1, alveolar type 1; AT2, alveolar type 2. Scale bars=50 μm.

FIG. 10. Transmission electron microscopy of gene-edited lungs of SftpcI73T/WT mice. Transmission electron microscopic (TEM) assessment of E19 lungs from SftpcI73T/WT mice injected with Ad.Sftpc.GFP or Ad.Null.GFP at E16 and uninjected C57BL/6 WT mice. Representative low magnification images show tufts of AT2 cells in Ad.Null.GFP injected SftpcI73T/WT mice and mature saccule formation in C57BL/6 WT mice and SftpcI73T/WT mice injected with Ad.Sftpc.GFP. Representative high magnification images demonstrate a hypertrophied AT2 cell with immature lamellar bodies and autophagosomes with double membranes (red arrowheads) in Ad.Null.GFP injected SftpcI73T/WT mice and mature lamellar bodies and release of surfactant vesicles (yellow arrowheads) into the alveolar lumen from AT2 cells in Ad.Sftpc.GFP injected SftpcI73T/WT mice and uninjected C57BL/6 WT mice. WT, wild-type; low magnification images, scale bar=10 μm; high magnification images, scale bar=2 μm.

FIGS. 11A-11G. Lung morphology of gene-edited Sftpc^{I73T/WT} mice in adulthood. (FIG. 11A) Schematic of the experimental design for long-term analysis of gene-edited Sftpc^{I73T/WT} mutant mice. (FIG. 11B) H and E staining of lungs from 13-week-old Sftpc^{I73T/WT} mice and C57BL/6 mice injected with Ad.Sftpc.GFP at E16 and uninjected WT C57BL/6 mice was performed to assess alveolarization. (FIG. 11C) The mean linear intercept was calculated in B to assess alveolarization. (FIG. 11D) IHC for SFTPB and HA was performed on lungs from 13-week-old Sftpc^{I73T/WT} mice and C57BL/6 mice injected with Ad.Sftpc.GFP at E16 and uninjected WT C57BL/6 mice to assess AT2 cell morphology and excision of the mutant Sftpc allele in AT2 cells. SFTPB⁺HA⁻ (yellow arrowheads indicate representative cells)=excision; SFTPB⁺HA⁺ (white arrowheads indicate representative cells)=no excision. (FIG. 11E) The percentage of SFTPB⁺HA⁻ cells, indicative of gene-edited cells in Ad.Sftpc.GFP injected Sftpc^{I73T/WT} mice, was quantified on IHC in D. (FIG. 11F) IHC for HOPX and AQP5 was performed to assess AT1 cell morphology in 13-week-old Sftpc^{I73T/WT} mice and C57BL/6 mice injected with Ad.Sftpc.GFP at E16 and uninjected WT C57BL/6 mice. (FIG. 11G) The internuclear distance was calculated in F to assess AT1 cell spreading. n=2-3 per group. IHC, immunohistochemistry; WT, wild-type; AT1, alveolar type 1; AT2, alveolar type 2. Scale bars=50 μm.

FIG. 12. Selection of sgRNAs for targeting of Sftpc gene and in vivo gene editing in Sheep model. Ovine SPC gene was screened in fetal sheep pulmonary cells and editing assessed by Surveyor assay.

### DETAILED DESCRIPTION OF THE INVENTION

Recent improvements in gene editing technology, including advances in CRISPR-Cas9 (Clustered Regularly Interspaced Short Palindromic Repeats [CRISPR]-CRISPR-associated 9) technology, offer an unprecedented opportunity for therapeutic correction of monogenic disorders (11-15). Standard CRISPR-Cas9 gene editing uses a single guide RNA (sgRNA) to instigate a double strand DNA break (DSB) in a site-specific fashion. Normal cellular mechanisms repair the DSB via nonhomologous-end joining (NHEJ), or if a donor repair template is provided, homology-directed repair (HDR) can be accomplished at low efficiency. Studies in postnatal mouse models have demonstrated the therapeutic potential of in vivo CRISPR-Cas9 gene editing to correct monogenic diseases (16-21).

Although postnatal in vivo gene editing studies are encouraging, some diseases, such as SP syndromes, result in morbidity and mortality at the time of or shortly after birth, precluding a postnatal approach. Several examples of early zygote gene editing have been described and could prove useful where mutation detection at very early developmental time points is possible (22-24). However, de novo mutations that occur later in development may not be treatable by early zygote gene editing. Using CRISPR-Cas9 gene editing to correct lung diseases during later prenatal developmental stages has the potential to reverse such genetic abnormalities before transition to postnatal life when pulmonary function becomes essential. In utero gene editing also provides the opportunity to take advantage of the normal developmental properties of the fetus to accomplish efficient gene editing. Specifically, the small size and immunologic immaturity of the fetus allow for the optimization of the CRISPR-Cas9 “dose” per recipient weight while avoiding a potential immune response to the bacterial Cas9 protein or delivering viral vector (25-28). Additionally, the target cell population for gene editing may be more accessible in the fetus. In the postnatal lung, immune and physical barriers including mucus and glycocalyx proteins limit access to pulmonary epithelial cells including alveolar type 2 (AT2) cells, the target cell population for SP disorders (29, 30). These immune and physical barriers are not as significant in the fetus, and multiple murine studies have demonstrated efficient gene transfer to pulmonary epithelial cells following prenatal viral vector delivery via intra-amniotic injection to take advantage of fetal breathing movements for lung targeting (31-33).

Monogenic lung diseases that are caused by mutations in surfactant genes of the pulmonary epithelium are marked by perinatal lethal respiratory failure or chronic diffuse parenchymal lung disease with few therapeutic options. Using a unique CRISPR fluorescent reporter system, we demonstrate that precisely timed in utero intra-amniotic delivery of CRISPR-Cas9 gene editing reagents during fetal development results in targeted and specific gene editing in fetal lungs. Pulmonary epithelial cells are predominantly targeted in this approach, with alveolar type 1, alveolar type 2, and airway secretory cells exhibiting high and persistent gene editing. We then used this in utero technique to evaluate a therapeutic approach to reduce the severity of the lethal interstitial lung disease observed in a mouse model of the human SFTPC^{I73T} mutation. Embryonic expression of Sftpc^{I73T} alleles is characterized by severe diffuse parenchymal lung damage and rapid demise of mutant mice at birth. Following in utero CRISPR-Cas9 mediated inactivation of the mutant Sftpc^{I73T} gene, fetuses and postnatal mice showed improved lung morphology and increased survival. These studies demonstrate that in utero gene editing is a novel and promising approach for treatment and rescue of monogenic lung diseases that are lethal at birth in animals and humans.

### I. Definitions

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non nucleotide components. A polynucleotide may be further modified after polymerization, such as by conjugation with a labeling component.

As used herein the term “wild type” is a term of the art understood by skilled persons and means the typical form of an organism, strain, gene or characteristic as it occurs in nature as distinguished from mutant or variant forms. As used herein the term “variant” should be taken to mean the exhibition of qualities that have a pattern that deviates from the wild type or a comprises non naturally occurring components.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%. 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

Several aspects of the invention relate to vector systems comprising one or more vectors, or vectors as such. Vectors can be designed for expression of CRISPR transcripts (e.g. nucleic acid transcripts, proteins, or enzymes) in prokaryotic or eukaryotic cells. For example, CRISPR transcripts can be expressed in bacterial cells such as Escherichia coli, insect cells (using baculovirus expression vectors), yeast cells, or mammalian cells. Suitable host cells are discussed further in Goeddel, GENE EXPRESSION TECHNOLOGY: METHODS IN ENZYMOLOGY 185, Academic Press. San Diego, Calif. (1990). Alternatively, the recombinant expression vector can be transcribed and translated in vitro, for example using T7 promoter regulatory sequences and T7 polymerase.

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter, U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the invention, an exogenous template polynucleotide may be referred to as an editing template. In an aspect of the invention the recombination is homologous recombination.

In some embodiments, one or more vectors driving expression of one or more elements of a CRISPR system are introduced into a host cell such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector comprises one or more insertion sites, such as a restriction endonuclease recognition sequence (also referred to as a “cloning site”). In some embodiments, one or more insertion sites (e.g. about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of a CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to a cell.

In some embodiments, a vector comprises a regulatory element operably linked to an enzyme-coding sequence encoding a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In aspects of the invention, nickases may be used for genome editing via homologous recombination.

In some embodiments, an enzyme coding sequence encoding a CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence.

In some embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions. Additional domains that may form part of a fusion protein comprising a CRISPR enzyme are described in US20110059502, incorporated herein by reference. In some embodiments, a tagged CRISPR enzyme is used to identify the location of a target sequence.

In an aspect of the invention, a reporter gene which includes but is not limited to glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into a cell to encode a gene product which serves as a marker by which to measure the alteration or modification of expression of the gene product. In a further embodiment of the invention, the DNA molecule encoding the gene product may be introduced into the cell via a vector. In a preferred embodiment of the invention the gene product is luciferase. In a further embodiment of the invention the expression of the gene product is decreased.

In some aspects, the invention provides methods comprising delivering one or more polynucleotides, such as or one or more vectors as described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a CRISPR enzyme in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line.

In one aspect, the invention provides for methods of modifying a target polynucleotide in a eukaryotic cell, which may be in vivo, ex vivo or in vitro. In some embodiments, the method comprises sampling a cell or population of cells from a human or non-human animal, and modifying the cell or cells. Culturing may occur at any stage ex vivo. The cell or cells may be re-introduced into the human or non-human animal.

In one aspect, the invention provides for methods of modifying a target polynucleotide in a eukaryotic cell. In some embodiments, the method comprises allowing a CRISPR complex to bind to the target polynucleotide to effect cleavage of said target polynucleotide thereby modifying the target polynucleotide, wherein the CRISPR complex comprises a CRISPR enzyme complexed with a guide sequence hybridized to a target sequence within said target polynucleotide, wherein said guide sequence is linked to a tracr mate sequence which in turn hybridizes to a tracr sequence.

In one aspect, the invention provides kits containing any one or more of the elements disclosed in the above methods and compositions. In some embodiments, the kit comprises a vector system or components for an alternative delivery system such as those described above and instructions for using the kit. In some embodiments, the vector or delivery system comprises (a) a first regulatory element operably linked to a tracr mate sequence and one or more insertion sites for inserting a guide sequence upstream of the tracr mate sequence, wherein when expressed, the guide sequence directs sequence-specific binding of a CRISPR complex to a target sequence in a eukaryotic cell, wherein the CRISPR complex comprises a CRISPR enzyme complexed with (1) the guide sequence that is hybridized to the target sequence, and (2) the tracr mate sequence that is hybridized to the tracr sequence; and/or (b) a second regulatory element operably linked to an enzyme-coding sequence encoding said CRISPR enzyme comprising a nuclear localization sequence. Elements may be provided individually or in combinations, and may be provided in any suitable container, such as a vial, a bottle, or a tube. In some embodiments, the kit includes instructions in one or more languages, for example in more than one language.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide.

In one aspect, the invention provides methods for using one or more elements of a CRISPR system. The CRISPR complex of the invention provides an effective means for modifying a target polynucleotide. The CRISPR complex of the invention has a wide variety of utility including modifying (e.g., deleting, inserting, translocating, inactivating, activating) a target polynucleotide in a multiplicity of cell types in methods of gene therapy. An exemplary CRISPR complex comprises a CRISPR enzyme complexed with a guide sequence hybridized to a target sequence within the target polynucleotide. The guide sequence is linked to a tracr mate sequence, which in turn hybridizes to a tracr sequence.

The materials and methods are provided below to facilitate the practice of the present invention.

### Methods

The feasibility and efficiency of prenatal lung gene editing after intra-amniotic delivery of CRISPR-Cas9 via adenoviral vector was evaluated in order to demonstrate that prenatal pulmonary gene editing can alter the phenotype of a perinatal lethal monogenic lung disease. Experimental animals were fetuses injected with viral vectors containing SpyCas9 and an sgRNA. Control animals were fetuses injected with viral vectors containing SpyCas9 and no sgRNA, Cre recombinase, or noninjected fetuses. Sample size was determined by availability and previous experience with in utero gene and cellular therapy experiments in the mouse model. No outliers were excluded from the study. A minimum of 3 animals per group were used for studies involving statistical analyses, and the n for individual experiments is indicated in the figure legends. Pregnant mice were randomly allocated to experimental and control groups. Intra-amniotic injections and dissections were conducted in a non-blinded fashion. Blinding was performed during data collection and analysis, when possible given the survival and morphology differences in treated and untreated groups. For each experiment, sample size reflects the number of independent biological replicates.

Selection of Single Guide RNAs (sgRNAs)

sgRNAs for the R26^{mTmG/+} and Sftpc^{I73T} mouse models were chosen based on high on-target efficiency and low off-target effects using the online tool at crispr.mit.edu (12). For R26^{mTmG/+} mouse experiments, sgRNAs were designed to target both the loxP sites flanking the mT-tdTomato and stop cassette, causing the edited cells to express EGFP. For Sftpc^{I73T} mouse experiments, sgRNAs were designed to target the 5′ and 3′ ends of Sftpc gene. The sgRNAs targeting the Sftpc gene were screened by Surveyor assay in vitro. Briefly, the Sftpc sgRNAs were cloned into plasmid pSpyCas9(BB)-2A-GFP (PX458; a gift from Feng Zhang; Addgene plasmid #48138) (12), which was used to transfect mouse Neuro-2a cells (N2a). Genomic DNA was extracted using DNeasy blood and tissue kit (QIAGEN) 48 hours after transfection. Indel efficiency of each sgRNA was assessed by Surveyor nuclease assay (IDT) as previously described after amplifying with primers flanking the target site (12). The protospacer and PAM sequences screened and the PCR primers used in the Surveyor assay are listed in tables 1 and 2.

### Generation of Adenovirus Vectors

The mTmG sgRNA was cloned into plasmid pX330-U6-Chimeric_BB-CBh-hSpyCas9 (a gift from Feng Zhang; Addgene plasmid #42230) (50). The Sftpc sgRNAs (1A-targeting exon 1 and 5B-targeting exon 5) which were noted to have activity on Surveyor assay during in vitro screening were used for the in vivo experiments. The plasmids pX330-U6-Chimeric_BB-CBh-hSpyCas9 and pSpyCas9(BB)-2A-GFP (PX458) in which no sgRNA was cloned served as the negative control for the R26^{mTmG/+} and Sftpc^{I73T} mouse experiments, respectively. Vector Biolabs used these constructs to generate recombinant adenovirus type 5 particles. Premade adenovirus (Ad) type 5 particles containing Cre recombinase under a CMV promoter were purchased from Penn Vector Core. Ad viral vectors are referred to as Ad.mTmG, Ad.Sftpc.GFP, Ad.Cre, Ad.Null, and Ad.Null.GFP. The final viral titer used for experiments ranged from 0.6×10¹⁰-1.2×10¹¹ PFU/ml.

### Animals

C57Bl/6, B6.129(Cg)-Gt(ROSA)26Sor^{tm4(ACTB-tdTomato,-EGFP)Luo}/J (called R26^{mTmG/+}; stock #007676), and B6.129S4-Gt(ROSA)26Sortm2(FLP*)Sor/J (called Flp-O mice; stock #12930) were purchased from Jackson Laboratories. Sftpc^{I73T} mice were created and provided by Dr. Michael Beers (35). Animals were housed in the Laboratory Animal Facility of the Abramson Research Center and the Colket Translational Research Building at The Children's Hospital of Philadelphia (CHOP). The experimental protocols were approved by the Institutional Animal Care and Use Committee at CHOP and followed guidelines set forth in the National Institutes of Health's Guide for the Care and Use of Laboratory Animals.

### In Utero Injection

Intra-amniotic in utero injections were performed as previously described (data not shown) (32). Briefly, the amniotic cavity of fetuses of time-dated mice was injected at gestational day (E) 16, a time during murine fetal development at which fetal breathing movements are optimal. Under isoflurane anesthesia and after providing local anesthetic (0.25% bupivacaine subcutaneously), a midline laparotomy was made and the uterine horn exposed. Under a dissecting microscope, 10 μL of virus combined with 10 μL of theophylline (1.6 mg/ml) were injected into the amniotic sac of each fetus. The uterus was then returned to the abdominal cavity and the laparotomy incision was closed in a single layer with 4-0 Vicryl suture. After recovery from anesthesia, pregnant dams were placed in a chamber containing 10% CO₂ for 1 hour. Theophylline injection and maternal CO₂ exposure was performed to enhance fetal respiratory drive to more efficiently target the fetal lung (40).

### R26^{mTmG} CRISPR Gene Editing Model

The Gt(ROSA)26Sor^{tm4(ACTB-tdTomato,-EGFP)Luo} (R26^{mTmG/+}) mouse model is a fluorescent reporter mouse model that consists of a membrane bound tdTomato (mT) and 3′ stop codon that is flanked by loxP sites. Downstream to the distal lox P site, is the membrane-bound green fluorescent protein (mG-EGFP). All cells at baseline express tdTomato. Expression of Cre recombinase causes deletion of the mT-tdTomato cDNA along with a transcriptional stop cassette and expression of the mG-EGFP (34). R26^{mTmG/+} fetuses were injected intra-amniotically with either Ad.mTmG, Ad.Cre, or Ad.Null at E16, and the injected fetuses were analyzed at E19, postnatal day 7 (P7), P30, and 6 months of age. At the time of analysis, the lungs and other organs of injected mice were assessed for EGFP expression by fluorescent stereomicroscope (MZ16FA; Leica). For larger mice at P30—6 months of age, the right lung was fixed in 2% paraformaldehyde for immunohistochemistry (IHC) analysis, and the left lung was used to extract genomic DNA with DNeasy blood and tissue kit (QIAGEN) and for fluorescence-activated cell sorting (FACS). At E19 and P7, the right lung was used for IHC and the left lung was used to extract DNA from a single mouse. Both the right and left lungs from another mouse were used for FACS. For the experimental group, all fetuses from a single dam were injected with Ad.mTmG. Ad.Cre was injected at comparable titers to all fetuses from another dam as a positive control, and Ad.Null without a sgRNA was injected at comparable titers for negative control experiments.

### Sftpc^{I73T} Mutant Mouse Model

The Sftpc^{I73T} mouse line has targeted alleles containing an HA-tagged mouse SP-C^{I3T} sequence knocked into the endogenous mouse Sftpc locus. Heterozygous mutant mice accumulate mistrafficked mutant SP-C^{3T} pro-protein within AT2 cells, causing arrest of lung morphogenesis and death within 6 hours of birth. An intronic FRT-PGK-neo-FRT cassette results in a homomorphous phenotype and enables mice to survive to adulthood (35). In this study, Sftpc^{I73T/I73T/Neo+/+} mice were crossed with FlpO^{+/+} mice to produce Sftpc^{I73T} fetuses. Two Ad-5 vectors with one virus expressing spyCas9, a sgRNA targeting the 5′ end of Sftpc gene, and EGFP and the other virus expressing spyCas9, a sgRNA targeting the 3′ end of Sftpc gene, and EGFP were injected into E16 Sftpc^{I73T/WT} or C57BL/6 fetuses. Fetuses were harvested at E19 for analysis as described above. For survival analysis of Sftpc^{I73T/WT} injected fetuses, pups were allowed to be born and fostered with Balb/c dams until P7, at which time they were euthanized by decapitation for morphological and IHC analysis.

### Gene Editing Assessed by PCR Analysis

Primers 5′ and 3′ to the sgRNA target sites in the mTmG-loxP and Sftpc gene were used for PCR analysis to detect gene editing (table 3). PCR amplification with mTmG primers results in a 2951 bp band for the unedited mTmG sequence and a 545 bp band for the edited mTmG sequence. Similarly, PCR amplification with Sftpc primers results in a 3828 bp band for the unedited Sftpc^{WT} gene, a 3950 bp band for the unedited Sftpc^{I73T} gene, and a 605 bp band for the edited Sftpc gene. For quantification of gene-edited Sftpc alleles, quantitative real-time PCR was performed on a QuantiStudio 7 Flex using SYBR green reagents and primers specific for the unedited and edited alleles (table 4).

### Lung Cell Isolation and Flow Cytometry

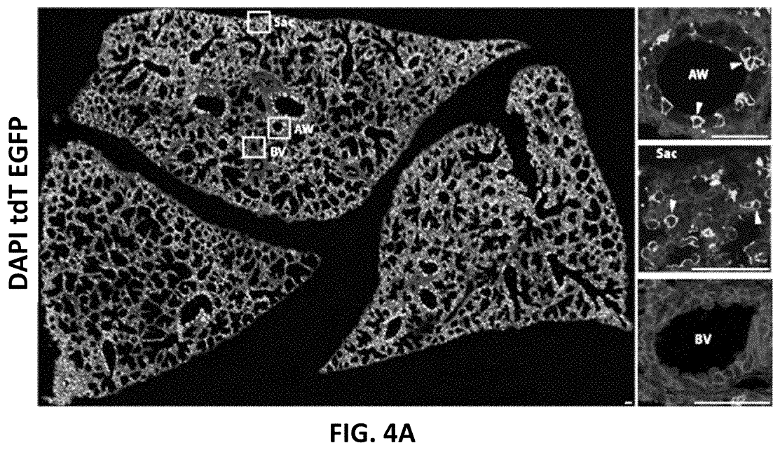

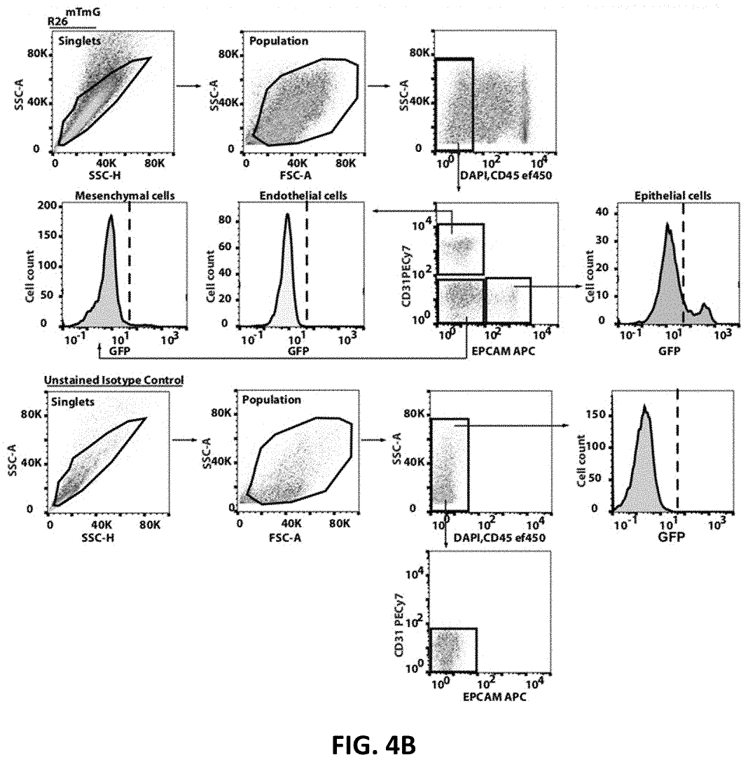

Lungs were harvested and processed into single-cell suspension using a dispase (Collaborative Biosciences)/collagenase (Life Technologies)/DNase solution as previously described (51). For the R26^{mTmG} experiments, lung epithelial, endothelial, and mesenchymal cell populations were assessed using a MoFlo Astrios EQ (Beckman Coulter) flow cytometer with antibody staining for DAPI, EpCAM-APC (eBioscience), CD31-PECy7 (eBioscience), and CD45-ef450 (eBioscience). Cells were negatively gated for DAPI and CD45 channels to exclude dead cells and lymphohematopoietic cells. Pulmonary epithelial (EpCAM⁺CD31⁻), endothelial (EpCAM⁻CD31⁺), and mesenchymal (EpCAM⁻CD31⁻) cells were evaluated for EGFP expression to determine the percentage of editing within each cell type (FIGS. 4E and 4F). Individual cell types were FACS-sorted, and DNA was extracted for PCR analysis as described above. Similarly, for the Sftpc^{I73T} mouse experiments, lung epithelial cell populations were sorted from the single-cell suspension using a MoFlo Astrios EQ (Beckman Coulter) flow cytometer with antibody staining for DAPI, EpCAM-APC (eBioscience), and CD45-PECy7 (eBioscience) and negatively gated for DAPI and CD45. The percentage of pulmonary epithelial cells transduced by adenovirus was measured by the percentage of Epcam⁺ cells that were EGFP⁺. Epcam⁺ cells were sorted and DNA was extracted for PCR analysis.

### Histology and IHC

Lungs were directly fixed in 2% paraformaldehyde. Lungs that were harvested for morphological analyses were inflation-fixed with 20 cm H₂O at E19 and 30 cm H₂O at P7 or later. After serial dehydration, tissue was embedded in paraffin and sectioned. Hematoxylin and eosin staining was performed for tissue morphology. IHC to detect proteins was performed using the following antibodies on paraffin sections: GFP (goat, Abcam, 1:100), GFP (chicken, Ayes, 1:500), RFP (rabbit, Rockland, 1:250), SFTPC (rabbit, Santa Cruz, 1:250), SFTPB (rabbit, Abcam 1:500), AQP5 (rabbit, Abcam, 1:100), SCGB1A1 (goat, Santa Cruz, 1:20), FOXJ1 (mouse, Santa Cruz, 1:250), HA (mouse, Abcam, 1:4000); HOPX (mouse, Santa Cruz, 1:50).

### Quantification of Edited Cells by IHC

Confocal microscopy using a Leica TCS SP8 confocal scope was used to capture images. For each mouse, confocal z stack images were taken in 5 or 10 random airway and alveolar areas, respectively, and analyzed using ImageJ software. The specific cell types that were EGFP⁺ in the R26^{mTmG} experiments or HA⁺ in Sftpc^{I73T} experiments were manually counted using the Cell Counter plug-in for ImageJ.

### Quantification of Alveolarization and AT1 Cell Spreading

For quantification of mean linear intercept, 10 pictures for each sample were taken with a 40× objective lens for E19 lungs and at 20× for P7 and adult lungs. The images were viewed under a field of equally spaced horizontal lines using ImageJ, and MLI was calculated as the average of total length of lines divided by the total intercepts of alveolar septa from each lung. For quantification of AT1 cell spreading, 5 pictures from each lung sample were taken with a 40× objective, and average distance between HOPX-stained AT1 cells was measured using ImageJ as previously described (52).

### Off-Target Analysis

Off-target sites for Sftpc were predicted using CRISPOR (http://crispor.tefor.net), and the top twenty sites, as ranked by the CFD off-target score (41), were assessed by next-generation DNA sequencing at the Massachusetts General Hospital CCIB DNA Core (CRISPR Sequencing Service; https://dnacore.mgh.harvard.edu/new-cgi-bim.site/pages/crispr_sequencing_main.jsp). Please refer to tables 5 and 6 for the predicted off-target sites and the PCR primers used for off-target NGS analysis. The number of paired-end reads typically exceeded 50,000 per target site per sample. Off-target indel mutagenesis rates were determined as previously described (18).

### Statistical Analyses

At least three mice were used for experimental and control groups undergoing statistical analyses, with the n values indicated in the figure legends. All animals that inhaled the virus after intra-amniotic delivery, as represented by EGFP⁺ lungs, were included for the final analysis. Animals that had EGFP⁻ lungs were considered as technical failure and excluded from final analysis. All data points used in statistical analyses are represented as the mean±one standard deviation (SD). For histologic analyses, all data points were means of technical replicates and presented as percentages or means±one SD. A two-tailed Student's t-test was used for experiments involving the comparison of two groups in which data were normally distributed, as determined by the Shapiro-Wilk test of normality. A one-way ANOVA followed by Tukey's multiple comparison tests was used for statistical analyses of experiments involving the comparison of more than two groups. Survival analysis of gene-edited Sftpc^{I73T} mice was performed using survival proportions and by log-rank (Mantel-Cox) test for comparison of survival curves. P<0.05 was considered significant. Statistical analyses were performed with GraphPad Prism 7.

The following examples are provided to illustrate certain embodiments of the invention. They are not intended to limit the invention in any way.

### Example I

### Pulmonary Gene Editing Following Intra-Amniotic Delivery of CRISPR-Cas9

In the present example we demonstrate the feasibility, efficiency, and specificity of prenatal CRISPR-Cas9 mediated gene editing of the lung in two mouse models. We first developed a targeting strategy for a commercially-available fluorescent reporter mouse model (34), to demonstrate efficient and persistent gene editing, and found that our approach predominantly restricted gene editing to pulmonary epithelial cells following intra-amniotic delivery of CRISPR-Cas9 reagents. We then evaluated the therapeutic role of fetal lung gene editing utilizing a mouse model expressing a chILD-causing mutation, Sftpc^{I73T}(35). When expressed in mice during embryogenesis, the SP-C^{I73T} proprotein arrests lung development leading to rapid perinatal death. We show that CRISPR-Cas9 induced excision of the mutant Sftpc^{I73T} gene can rescue the lung from toxic accumulation of the disease-associated protein and improve lung development in Sftpc^{I73T} mutant mice, leading to their increased survival. Our proof-of-concept study demonstrates that in utero gene editing provides a new therapeutic approach for treatment of congenital lung diseases caused by defects in the pulmonary epithelium.

### Results

### Intra-Amniotic Delivery of CRISPR-Cas9 Results in Efficient Pulmonary Gene Editing

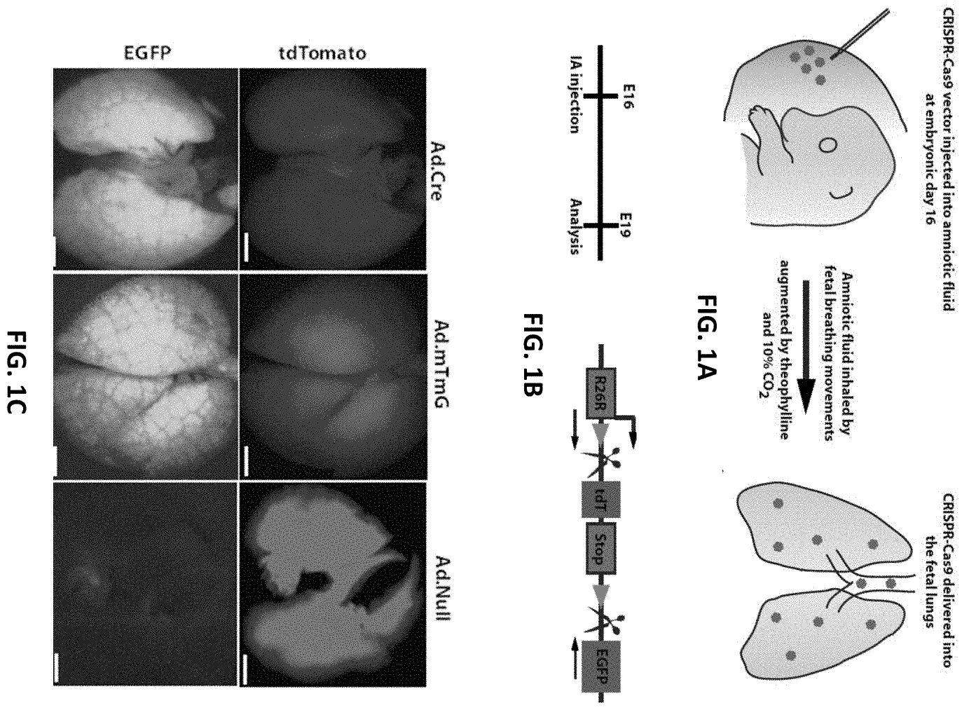

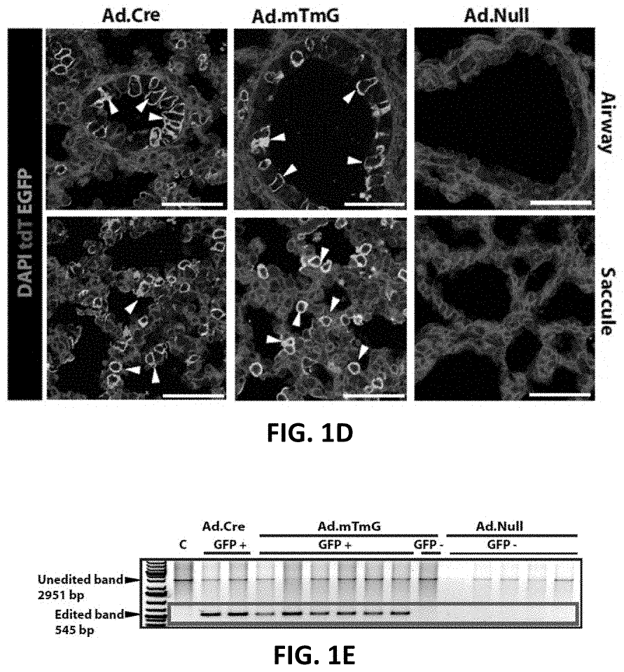

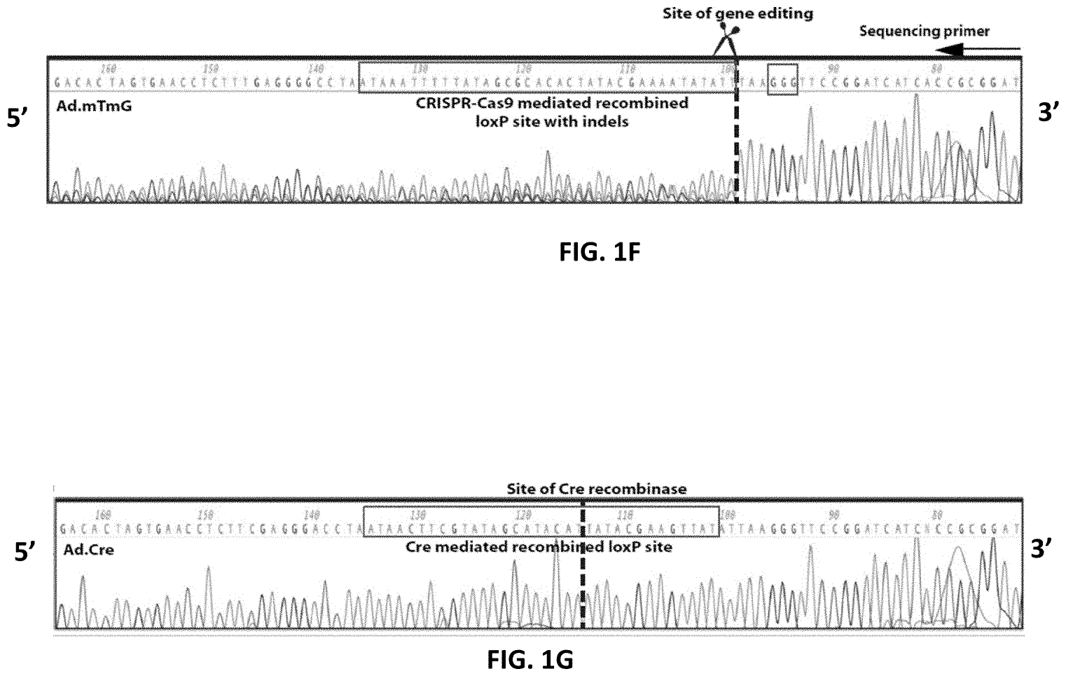

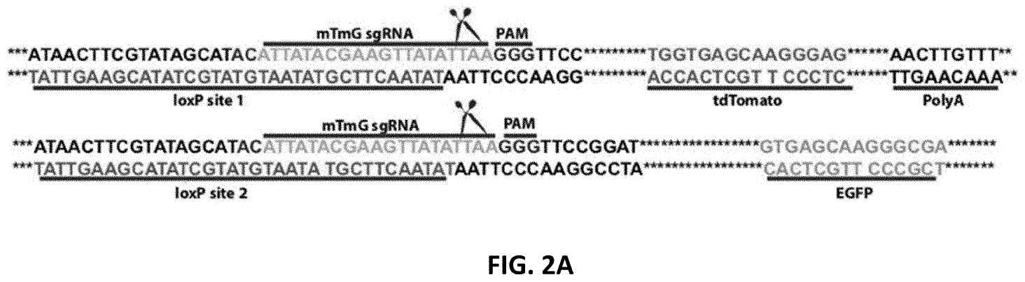

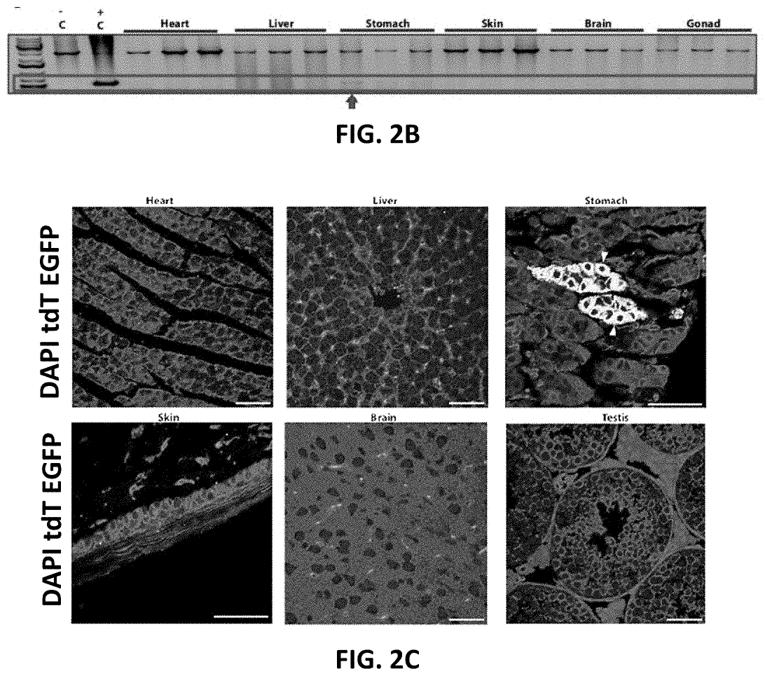

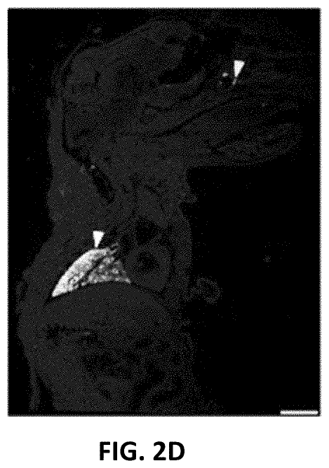

We established a model to define the efficiency and persistence of prenatal lung gene editing that could be easily monitored and quantified. Gt(ROSA)26Sor^{tm4(ACTB-tdTomato,-EGFP)Luo} mice (referred to as R26^{mTmG}) have a two-color fluorescent cassette (mT-tdTomato: cell membrane-bound red; mG-EGFP: cell membrane-bound green) that can be differentially activated by Cre recombinase (34). mT-tdTomato red fluorescence is constitutively expressed in the plasma membrane of all cells, including pulmonary epithelial, endothelial, and mesenchymal cells. Upon Cre expression, the mT-tdTomato cDNA along with a transcriptional stop cassette is deleted and the mG-EGFP cassette is subsequently expressed. We chose Streptococcus pyogenes Cas9 (SpyCas9) to perform in utero CRISPR-Cas9 gene editing because SpyCas9 remains the most efficient version of the enzyme. Due to the large size of SpyCas9 (˜4.2 kb), we chose an adenovirus (Ad) to deliver this enzyme to the developing fetus. As previous work has demonstrated that fetal breathing movements combined with theophylline and mild maternal hypercarbia treatment to stimulate respiratory drive can promote efficient and fairly specific delivery of viral vectors into the fetal lung after intra-amniotic injection (32, 33), we delivered Ad vectors containing SpyCas9 and a sgRNA targeting the loxP sites flanking the mT/stop cassette (Ad.mTmG) into the amniotic cavity of E16 R26^{mTmG/+} fetuses (FIG. 1A, FIG. 2A, table 1). Injected fetuses were assessed for editing at E19 (FIG. 1B). Control fetuses were injected with either an Ad vector containing Cre recombinase (Ad.Cre; positive control) or an Ad vector containing SpyCas9 and no sgRNA (Ad.Null). Fetuses injected with Ad.Cre and Ad.mTmG underwent extensive pulmonary gene editing, as supported by the presence of membrane-bound EGFP⁺ cells lining both the proximal airways and distal saccules In contrast, fetuses injected with Ad.Null lacked the expression of membrane-bound EGFP (FIG. 1C,D). PCR analysis of genomic DNA from lungs of injected fetuses supported efficient gene editing with excision of the mT/stop cassette and subsequent NHEJ using Ad.mTmG (FIG. 1E). DNA Sanger sequencing revealed that the edited sequence contained indels in the recombined loxP region beginning three nucleotides 5′ to the PAM site (FIG. 1F). In contrast, indels were absent in the Cre-mediated recombined loxP site (FIG. 1G). In addition to the lung, rare clusters of EGFP⁺ edited cells and PCR analysis consistent with editing were noted in the stomach, consistent with previous studies demonstrating transduction of the proximal gastrointestinal tract after intraamniotic Ad injection (33) (FIG. 2B,C). Gene editing was not detected by PCR or immunohistochemistry in the heart, liver, skin, brain, and gonads. These data are further supported by the restricted delivery and expression of the Ad vector to the lung at this developmental time point (FIG. 2D). Thus, intra-amniotic delivery of Ad vectors carrying CRISPR-Cas9 results in pulmonary gene editing.

TABLE 1
mTmg and Sftpc sgRNAs for in
vitro and in vivo editing

| Target | Protospacer | PAM | SEQ. ID # |
|---|---|---|---|
| mTmG | ATTATACGAAGTTATATTAA | GGG | SEQ. ID# 1 |
| Sftpc exon 1A | TCAGAGCCCAGGCCCCGATA | AGG | SEQ. ID# 2 |
| Sftpc exon 1B | CAGCTGCCTTATCGGGGCCT | GGG | SEQ. ID# 3 |
| Sftpc exon 1C | CTCTTGCAGCTGCCTTATCG | GGG | SEQ. ID# 4 |
| Sftpc exon 5A | AGGTGTCTCTCCTACGGGCC | AGG | SEQ. ID# 5 |
| Sftpc exon 5B | ATAGGATCCCCCTGGCCCGT | AGG | SEQ. ID# 6 |
| Sftpc exon 5C | GGTAGAAACCGCAGCGGGAC | AGG | SEQ. ID# 7 |
| Sftpc exon 5D | TACAGACTTCCACCGGTTTC | TGG | SEQ. ID# 8 |
| Sftpc exon 5E | ACAGGAAAGACCCTCCGCAA | AGG | SEQ. ID# 9 |

### Intra-Amniotic CRISPR-Cas9 Delivery Targets Pulmonary Epithelial Cells for Gene Editing

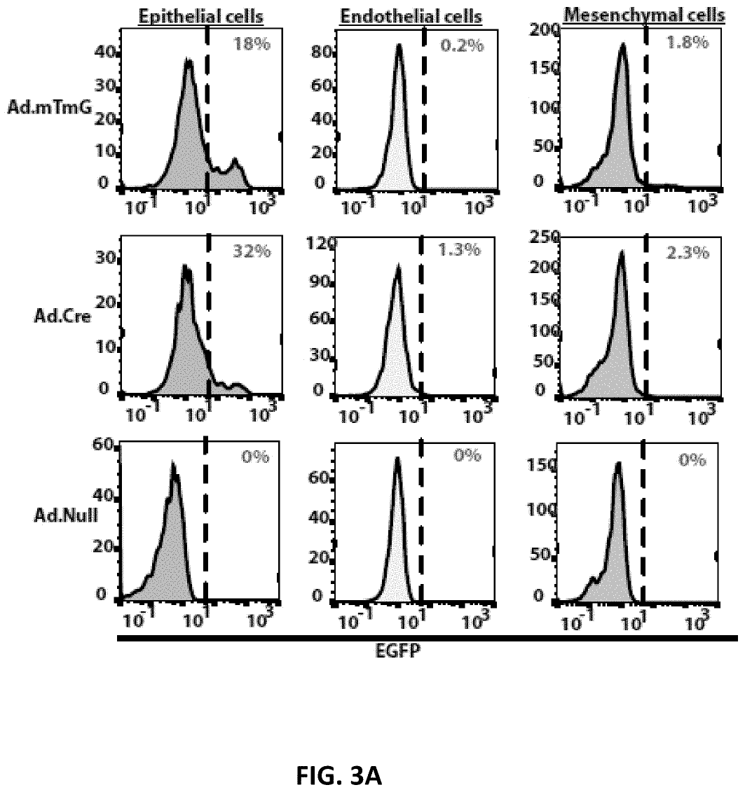

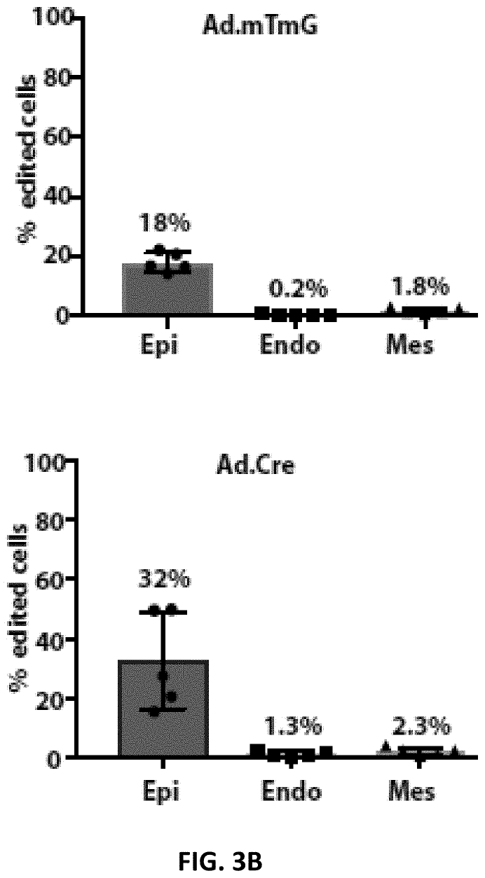

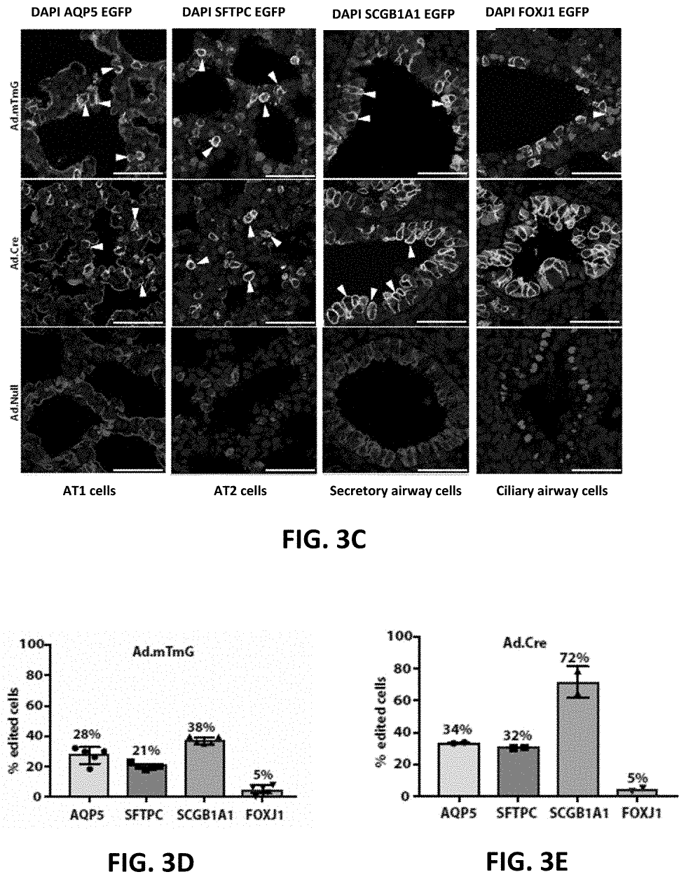

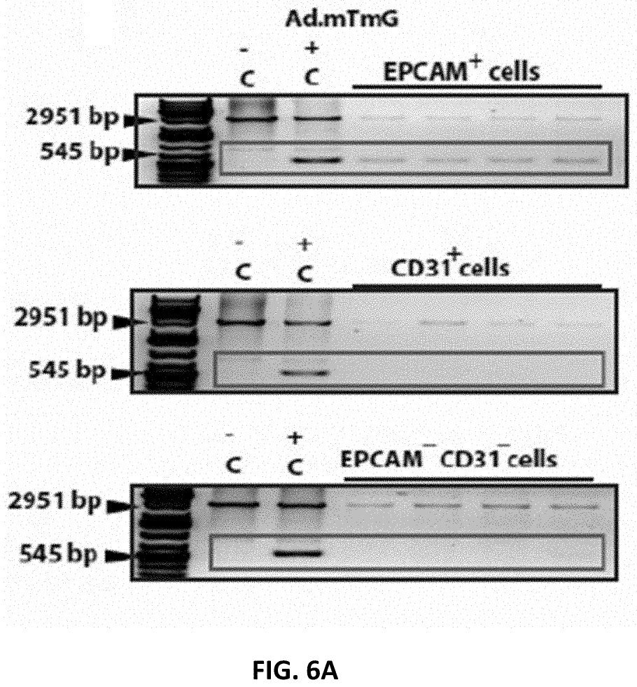

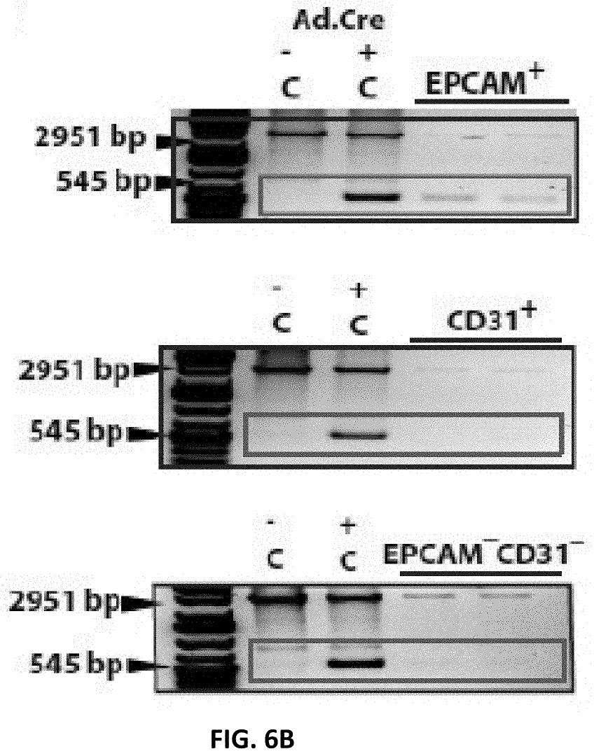

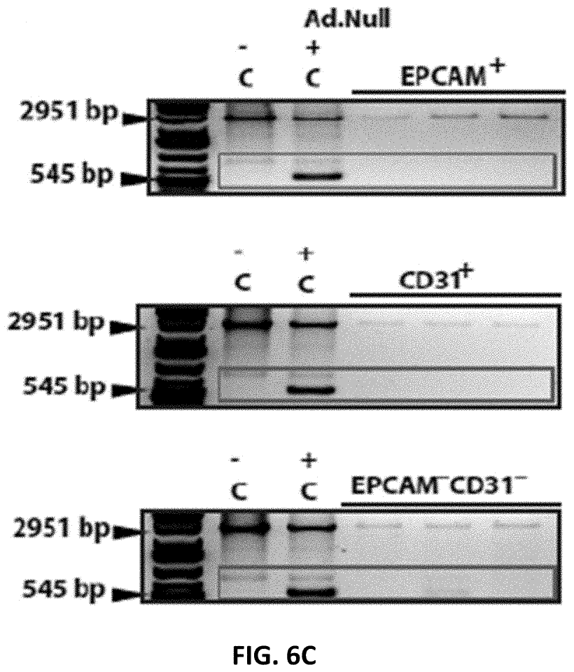

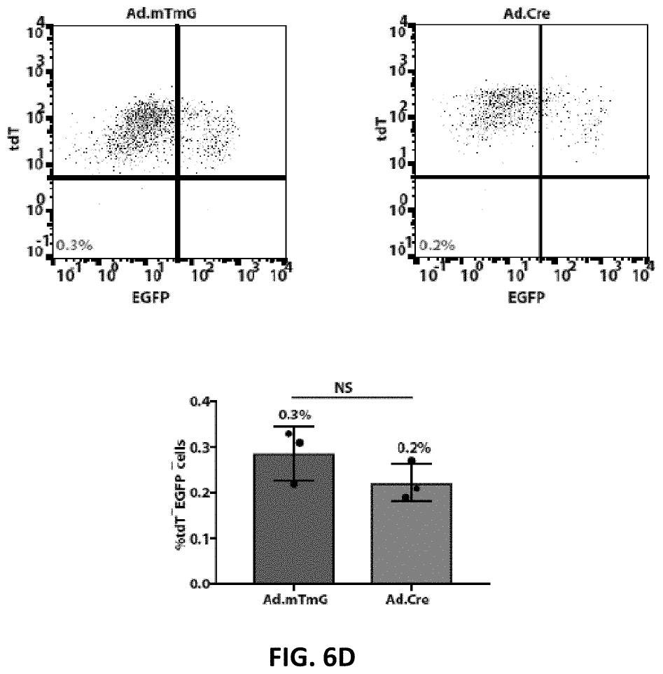

Since various lung epithelial cell types are affected in congenital monogenic lung diseases, we evaluated the efficiency of gene editing in individual pulmonary cell lineages (epithelial, endothelial, and mesenchymal cells) using the R26^{mTmG/+} model. The distribution of EGFP⁺ cells was confined to the epithelial lining throughout the lung section with sparing of the blood vessels and subepithelial regions (FIG. 4A). Using flow cytometry, we quantified the fraction of pulmonary epithelial (CD45⁻/DAPI⁻/EPCAM⁺), endothelial (CD45⁻/DAPI⁻/CD31⁺), and mesenchymal cells (CD45⁻/DAPI⁻/EPCAM⁻/CD31⁻) that were EGFP⁺, and thus edited, in the R26^{mTmG/+} model just before birth at E19 (FIG. 3A, FIG. 4B). EPCAM⁺ epithelial cells had the highest percentage (18%) of gene-edited cells (FIG. 3B). The Ad.Cre control showed a similar distribution of EGFP⁺ live pulmonary cell types. The percentage of EGFP⁺EPCAM⁺ cells was not significantly different in the Ad.Cre group compared to Ad.mTmG group (p=0.08) (FIG. 3B). To confirm the cell type-specific efficiency of gene editing in the lung, genomic DNA from fluorescence-activated cell sorting (FACS) of isolated epithelial, endothelial, and mesenchymal cells was assessed by PCR. Consistent with the flow cytometry data, the gene-edited 545 bp band was amplified in DNA from epithelial cells but not endothelial or mesenchymal cells from lungs of mice prenatally injected with Ad.mTmG or Ad.Cre (FIG. 6A-C).

We next assessed the efficiency of gene editing in several important lung epithelial lineages including AQP5⁺ alveolar epithelial type 1 cells (AT1), SFTPC⁺ type 2 alveolar epithelial cells (AT2), SCGB1A1⁺ secretory airway epithelial cells, and FOXJ1⁺ ciliated airway epithelial cells (FIG. 3C). This analysis demonstrated that gene editing occurred in all epithelial cell subpopulations including AT2 cells, the target cell population for SP disease and other congenital lung diseases (FIG. 3D). The distribution of gene-edited pulmonary epithelial cell subpopulations was similar to that seen in Ad.Cre-injected fetuses (FIG. 3C, 3E). Membrane-bound EGFP⁺ cells were not detected in Ad.Null-injected fetuses, pointing to the lack of spurious EGFP expression in nonedited cells and the specificity of this marker for gene editing in our model specificity of EGFP⁺ cells resulting from gene editing (FIG. 3C). Finally, a recent study suggests the possibility of large unwanted deletions or complex rearrangements after CRISPR-Cas9 gene editing (36). The R26^{mTmG/+} model provides an elegant system in which to assess for this event. Specifically, an unwanted large deletion at the R26^{mTmG} allele would likely inactivate both the mT and mG fluorescent reporters, resulting in EGFP⁻tdT⁻ cells. Analysis of EPCAM⁺ cells did not demonstrate an increase in the double negative cell population in the lungs of Ad.mTmG compared to Ad.Cre injected mice, suggesting that this event did not occur above background levels at a high frequency (FIG. 6D).

Pulmonary Epithelial Cell Editing is Stable after Prenatal CRISPR Delivery

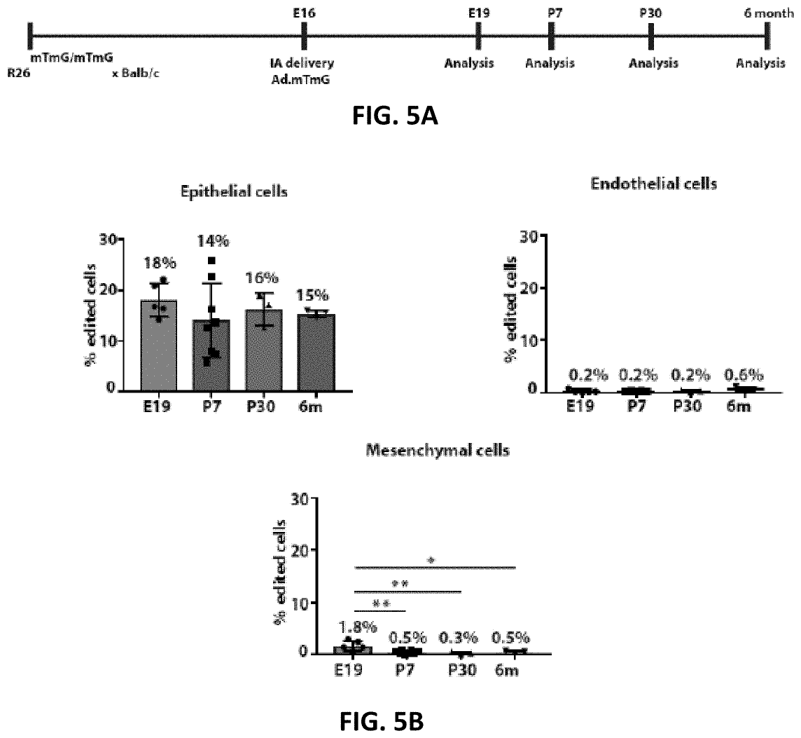

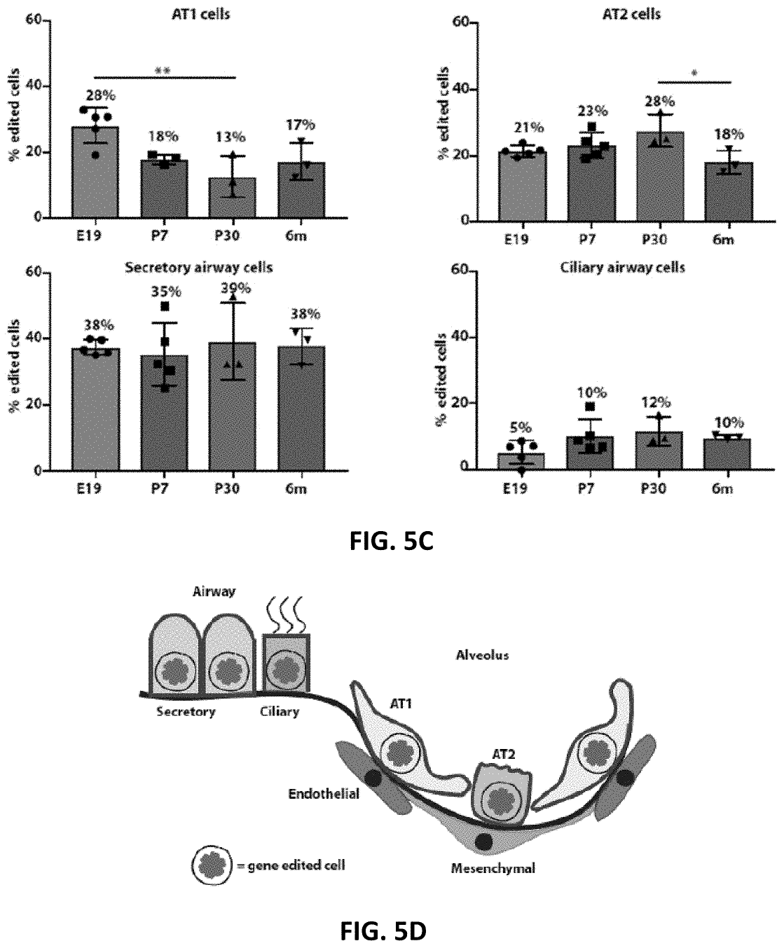

Although the lung is considered to be a fairly quiescent organ, there is a slow steady-state turnover of cells in the postnatal period. Using the R26^{mTmG/+} model, we assessed the persistence of gene-edited pulmonary cells over time using flow cytometry and IHC at gestational day (E) 19, postnatal day (P) 7, P30, and 6 months after intra-amniotic injection of Ad.mTmG at E16 (FIG. 5A). The percentage of gene-edited EGFP⁺ lung epithelial cells, the pulmonary cell lineage with highest gene editing efficiency, did not change over time, although there was a slight decrease in the number of gene-edited mesenchymal cells at later time points (FIG. 3B). We also assessed whether there were epithelial lineage-specific changes in the persistence of gene editing in the lung. The percentage of gene-edited secretory airway epithelial cells and ciliated airway epithelial cells remained stable over time, with only AT1 and AT2 cells demonstrating a slight decrease at P30 and 6 months, respectively (FIG. 5C). Thus, stable and highly specific gene editing is observed in most lung epithelial cells using the R26^{mTmG/+} model (FIG. 5D).

Prenatal Gene Editing in Sftpc^{I73}T Mice Decreases Mutant SP-C^{I73}T Proprotein and Improves Lung Alveolarization

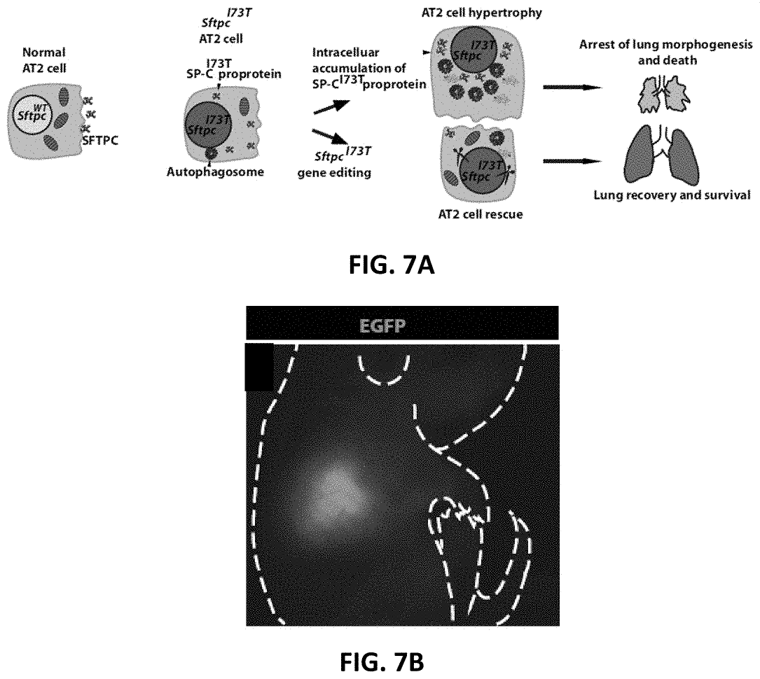

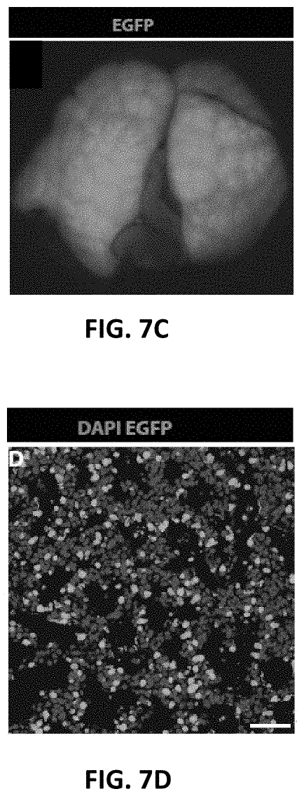

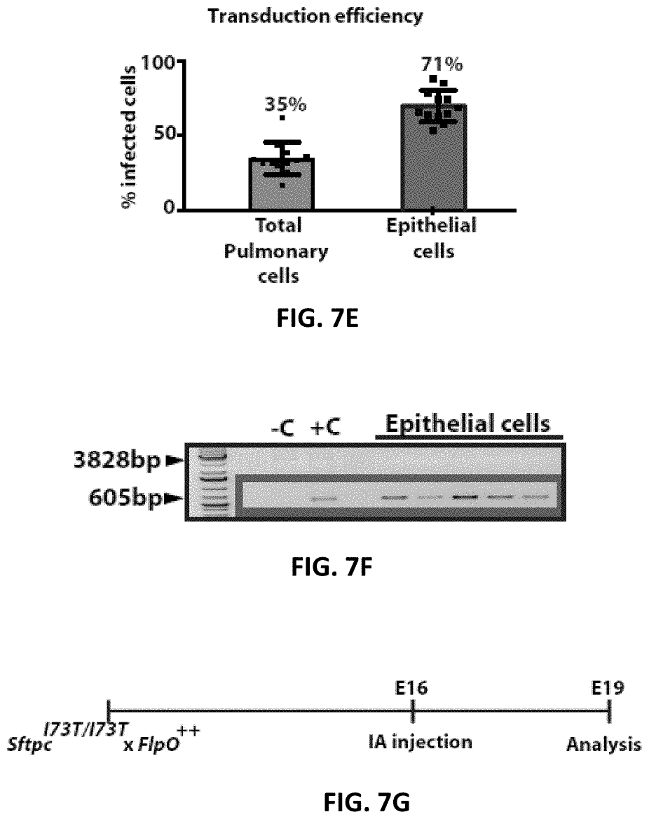

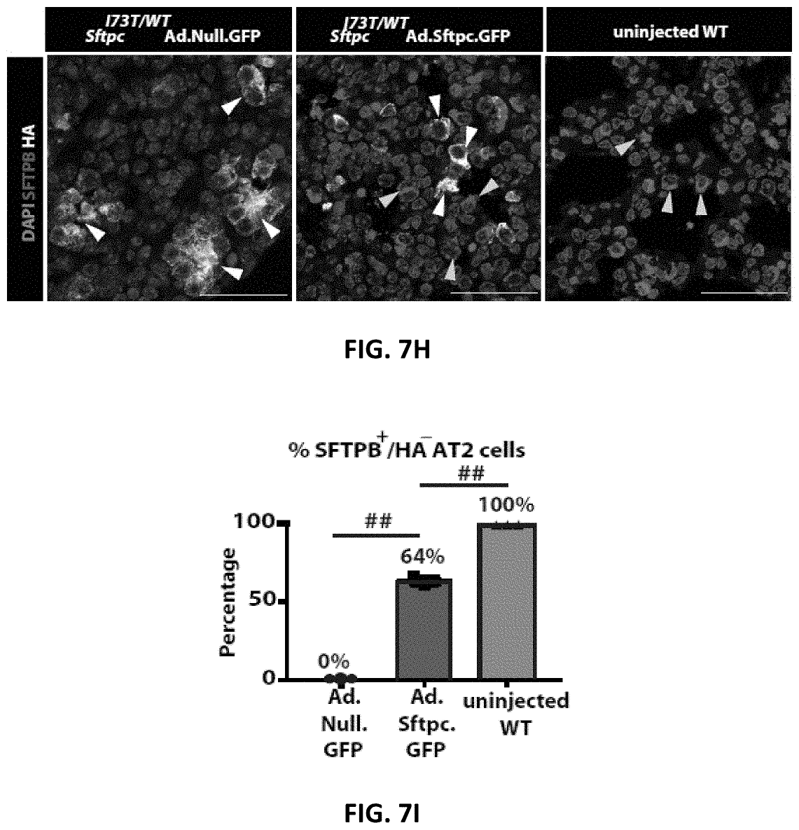

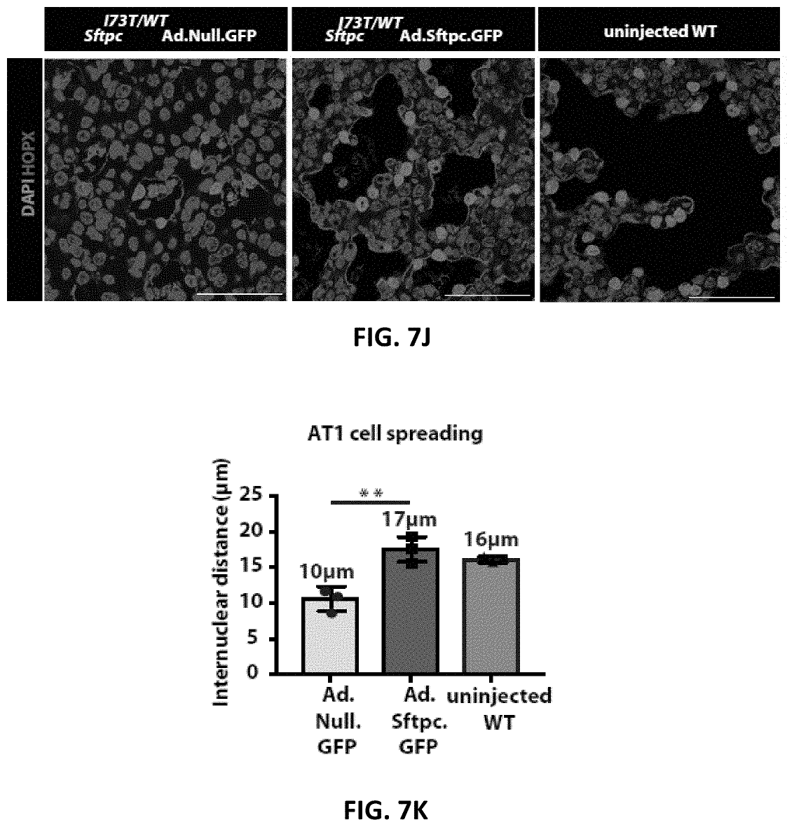

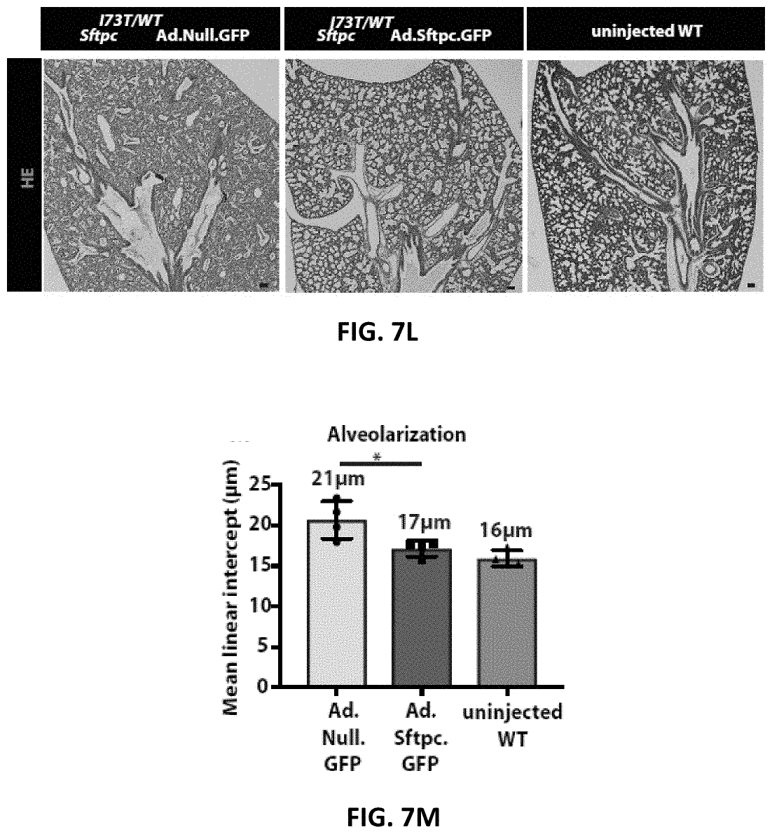

Because our data demonstrated effective and persistent gene editing in the developing lung, we next tested whether prenatal gene editing can rescue a clinically relevant monogenic human lung disease model. Among SFTPC variants associated with clinical ILD, the missense substitution (g.1286T>C), resulting in a change of isoleucine to threonine at position 73 in the SP-C proprotein (“SP-C^{I73T}”), is the most common known SFTPC mutation in humans (7, 37). Functionally, expression of SFTPC^{I73T} in vitro results in a toxic cellular response initiated by the markedly altered intracellular trafficking of the SP-C^{I73T} proprotein to the plasma membrane (38, 39). The Sftpc^{I73T} knock-in mouse, which models human SFTPC^{I73T}, has shown that intracellular accumulation of mutant proprotein triggers an aberrant injury-repair response resulting in fibrotic lung remodeling in adult mice (35). Under appropriate conditions, Sftpc^{I73T} mice can be induced to show an allele-dependent arrest of lung morphogenesis in late sacculation with no live births. Because previous studies have shown that Sftpc null mice have normal growth and lung function in homeostasis (40), we hypothesized that excision of the Sftpc^{I73T} gene would reduce the synthesis of mistrafficked SP-C^{I73T} proprotein, thereby correcting the dysfunctional AT2 cell phenotype and improving survival in gene-edited Sftpc^{I73T} mice (FIG. 7A).

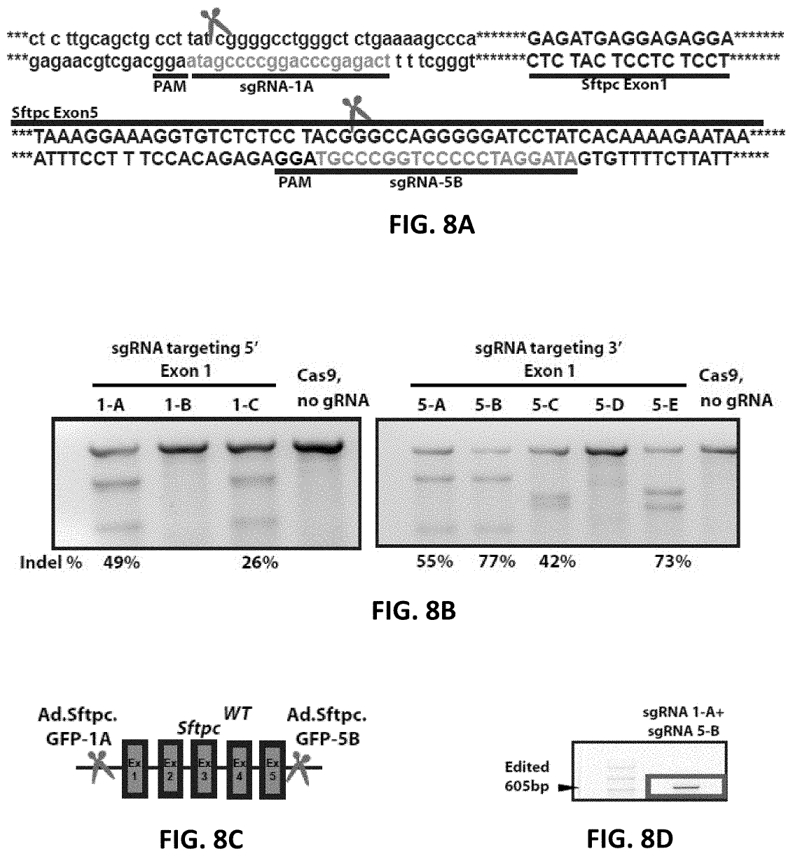

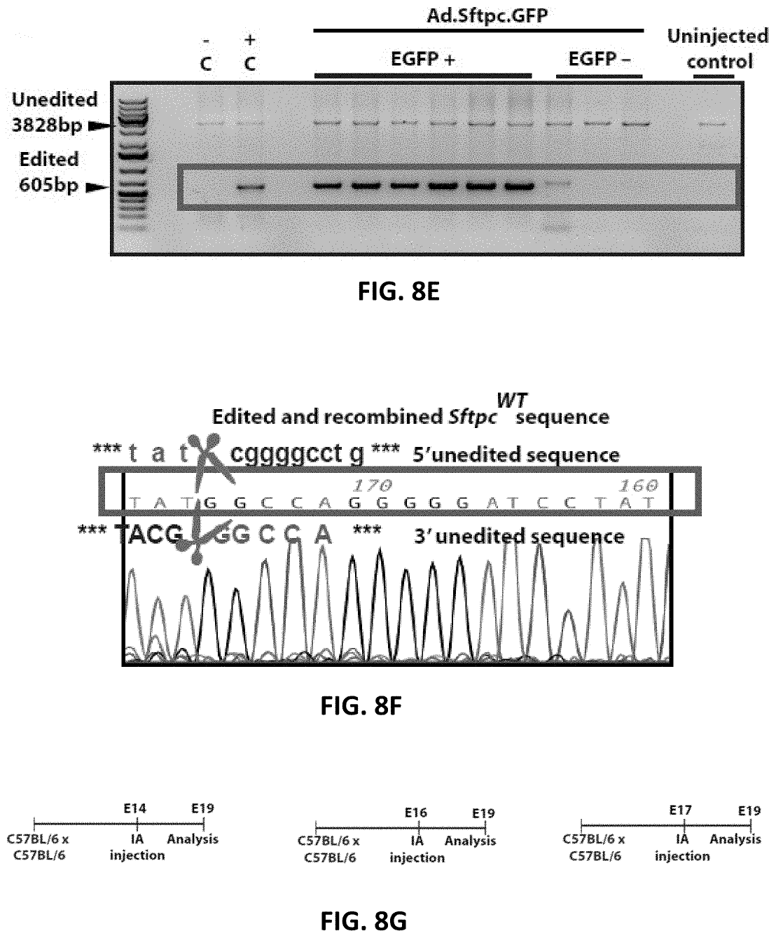

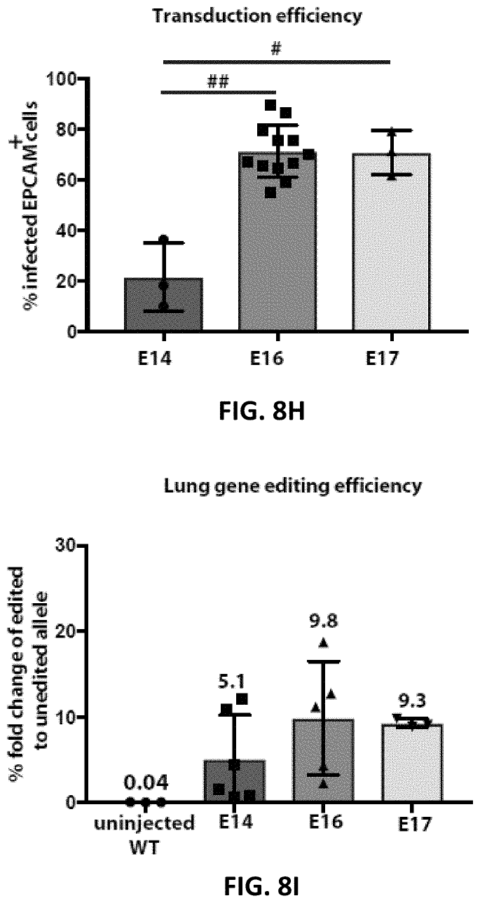

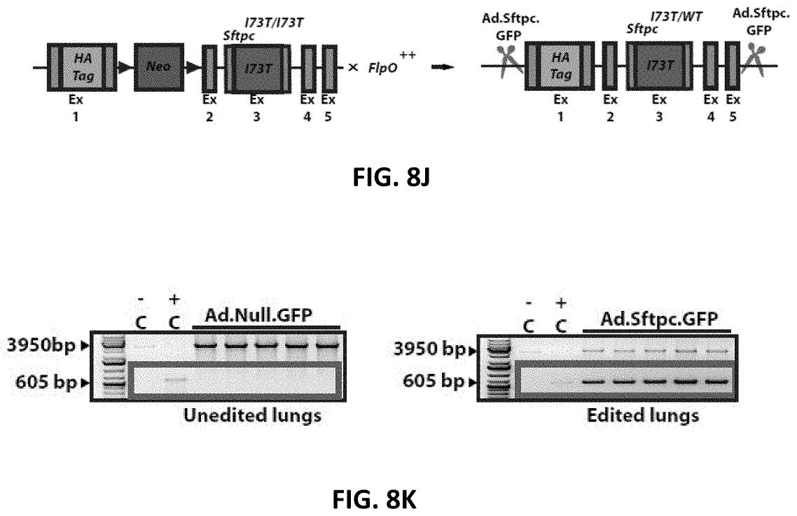

sgRNAs were designed to target the 5′ and 3′ ends of the Sftpc gene and screened for efficient DNA cutting (FIG. 8A-D, tables 1-2). Two Ad vectors containing SpyCas9 and EGFP cassette along with two of the selected sgRNAs (sgRNA 1A and 5B) for the Sftpc gene were used for intra-amniotic injections at E16 (collectively called the Ad.Sftpc.GFP). As predicted from the R26^{mTmG/+} model, injected wild-type C57BL/6 fetuses demonstrated EGFP fluorescence in the lungs (FIG. 7B-C), with EGFP⁺ cells lining the lung epithelium (FIG. 7D). As expected, a large proportion of CD45⁻/DAPI⁻/EPCAM⁺ cells were EGFP⁺ on flow cytometry analysis, supporting efficient transduction of pulmonary epithelial cells using Ad.Sftpc.GFP (FIG. 7E). To determine if CRISPR-mediated Sftpc excision and NHEJ occurred in fetal recipients of Ad.Sftpc.GFP, lung genomic DNA was assessed by PCR using primers flanking the Sftpc gene (table 3). The 605 bp band, corresponding to the excision of the Sftpc gene, was only present in fetuses that were EGFP⁺ and was faint or absent in the fetuses that were EGFP⁻ (FIG. 8E). Sanger sequencing confirmed editing and NHEJ at the expected sites (FIG. 8F). Analysis of FACS-sorted pulmonary epithelial cells from EGFP⁺ lungs also confirmed deletion of the Sftpc gene and NHEJ at the expected sites (FIG. 7F). Finally, to assess if pulmonary cell transduction and editing could be improved by altering the timing of intra-amniotic injection, fetuses were injected with Ad.Sftpc.GFP at E14 or E17 and results compared to those injected at E16 (FIG. 8G). At all time points, intra-amniotic delivery of Ad.Sftpc.GFP resulted in gene editing in the lung (FIGS. 8H and 8I, table 4). Given the desire to maximize both the editing efficiency and time between editing and birth, we elected to use the E16 time point for rescue experiments in the Sftpc^{I73T} mouse model.

TABLE 2
Primers used for surveyor assay for Sftpc gRNAs

| Target | Primer | | SEQ ID # |
|---|---|---|---|
| Sftpc | PCR | GCAGTCTGACCCTAAGGAAC | SEQ. ID# 10 |
| gRNA exon | forward | | |
| 1A-C | PCR | GGACTCTCCATCAGGACCTC | SEQ. ID# 11 |
| | reverse | | |
| Sftpc | PCR | GGAGGAAGGGCATGATACTG | SEQ. ID# 12 |
| gRNA | forward | | |
| 5A-E | PCR | TTGCTCTGTTCCCCATTACC | SEQ. ID# 13 |
| | reverse | | |

TABLE 3
Primers used for PCR and Sanger sequencing

| Target | primer | | SEQ ID# |
|---|---|---|---|
| mTmG | Sanger | CCTGTCCGTTCGCTTTGGAAG | SEQ. ID# 14 |
| | sequencing | | |
| mTmG | PCR | AAATCTGTGCGGAGCCGAAA | SEQ. ID# 15 |
| | forward | TC | |
| | PCR reverse | CCTGTCCGTTCGCTTTGGAAG | SEQ. ID# 16 |
| Sftpc | Sanger | TTGCTCTGTTCCCCATTACC | SEQ. ID# 17 |
| | sequencing | | |
| Sftpc | PCR | GCAGTCTGACCCTAAGGAAC | SEQ. ID# 18 |
| | forward | | |
| | PCR reverse | TTGCTCTGTTCCCCATTACC | SEQ. ID# 19 |

TABLE 4
Primers used for qPCR for Sftpc gene deletion

| Target | primer | | SEQ ID # |
|---|---|---|---|
| Sftpc | PCR forward | ACCCAGGTTTGCTCTTGTT | SEQ. ID# 20 |
| unexcised | PCR reverse | CTTGGCTTTGTAGCTTGTTTGT | SEQ. ID# 21 |
| Sftpc excised | PCR forward | GAGTTTGCTTACCTCACCCA | SEQ. ID# 22 |
| | PCR reverse | CCAACTCTCCAAACCCTCTC | SEQ. ID# 23 |

The founder Sftpc^{I73T-Neo} mouse line has a targeted allele containing an HA-tagged mouse Sftpc^{I73T} sequence knocked into the endogenous mouse Sftpc locus (35). This allele contains an intronic FRT flanked PGK/neo cassette producing a milder phenotype. Deletion of the neo cassette using a homozygous FlpO deleter line results in increased expression of Sftpc^{I73T} and a more severe phenotype characterized by abnormalities in sacculation, prenatal arrest of lung development, and perinatal death (FIG. 8J). E16 Sftpc^{I73T/WT} fetuses were injected with Ad.Null.GFP or Ad.Sftpc.GFP and harvested at E19 for analysis (FIG. 7G). EGFP⁺ lungs were examined for Sftpc gene editing by PCR analysis using primers flanking the sgRNA target sites. The smaller 605 bp Sftpc gene-edited band was detected in the Ad.Sftpc.GFP-injected mice but not control Ad.Null.GFP-injected mice (FIG. 8K). To quantify Sftpc gene editing, EGFP⁺ lungs were examined by IHC for co-expression of surfactant protein B (SFTPB), an AT2 cell marker, and the HA tag, which should be deleted after CRISPR mediated excision of the mutant allele. Whereas all the AT2 cells were HA⁺ in Ad.Null.GFP-injected fetuses, only 36% of the AT2 cells were HA⁺ in Ad.Sftpc.GFP-injected fetuses (FIGS. 7H and 7I). Importantly, Ad.Null.GFP-injected fetuses demonstrated clusters of HA⁺ AT2 cells within compressed and poorly formed saccules of the Sftpc^{I73T} mutant lungs, whereas Ad.Sftpc.GFP-injected lungs exhibited a greater number of normal-appearing saccules with AT2 cells showing a more typical punctate type of SFTPB staining, suggesting improved AT2 cell function. Furthermore, Ad.Sftpc.GFP-injected lungs also showed improved AT1 cell morphology as depicted by improved internuclear distance of HOPX-stained cells, signifying the characteristic cell spreading of AT1 cells (FIGS. 7J and 7K).

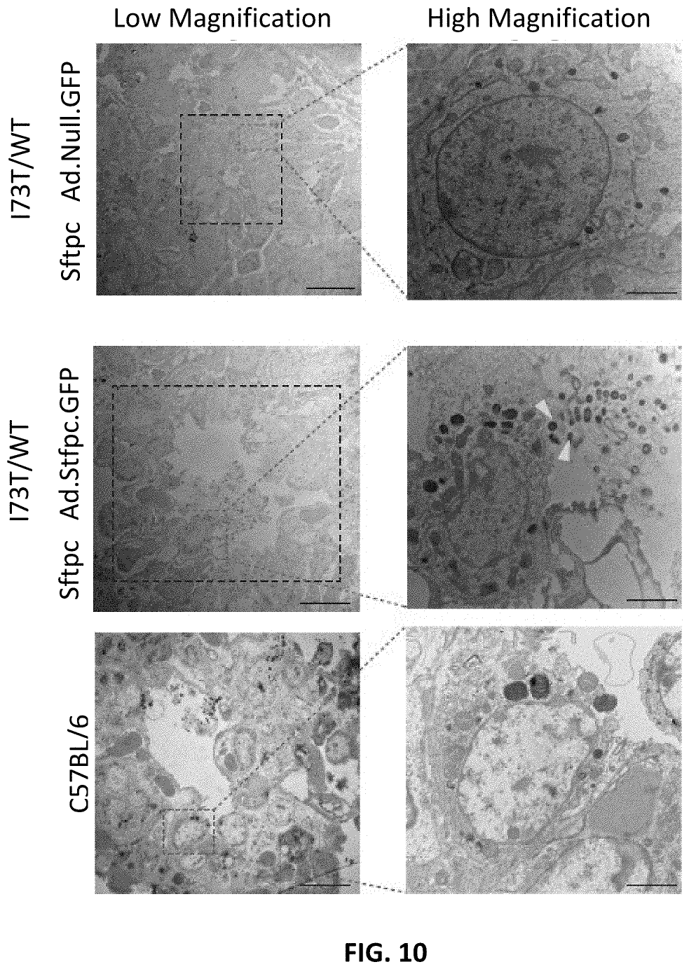

To assess improvement in lung alveolarization after rescue, lungs of E19 fetuses were inflation-fixed for morphometric analysis. Ad.Sftpc.GFP-treated mice demonstrated decreased mean linear intercept (MLI) compared to Ad.Null.GFP treated fetuses, indicating improved lung sacculation (FIGS. 7L and 7M). Further analysis by transmission electron microscopy revealed the presence of more mature AT2 cells with lamellar bodies and release of surfactant vesicles into the airspace lumen in Ad.Sftpc.GFP-injected mice, whereas the Ad.Null.GFP injected mice showed tufts of hypertrophied AT2 cells with excessive auto-phagosomes and immature lamellar bodies (FIG. 10). Next-generation sequencing (NGS) analysis of insertions and deletions (indels) from 20 top off-target sites as predicted by CRISPOR (41) in lung genomic DNA from Ad.Sftpc.GFP injected and uninjected Sftpc^{I73T/WT} fetuses showed that indel rates in experimental animals were equal to those seen in the control for all sites (Tables 5-6).

TABLE 5
Primer sequences for next-generation sequencing of Sftpc off-target sites

| Target | forward primer* | reverse primer* |
|---|---|---|
| intron:Ehd2 | TCTGTGTCTAGGACTATCCCAAAT (24) | GAGGGACGTCTGTCTCAGAA (25) |
| intron:Limk2 | TCCACACCCTTTAGGTCAATGC (26) | TATGCCAGGGATTTGGGCAC (27) |
| intron:Actn4 | CCTGTGGAGATAAGCAGGGC (28) | CTCTCTTGCCTCTCCTCCCT (29) |
| intron:Cars | AGACACCTAAGGAACAAGGCTG (30) | TCCAGTAAGGACAGCTGGGAC (31) |
| intergenic:Wnt9a- | CCTCTGCACAGAAGGTGCTT (32) | TAAGCTTCCAGCTGGCTTCC (33) |
| Prss38 | | |
| intron:Hs6st3 | GTCCCACAGACATTGATTCTCA (34) | AATCTAGATCTGCCTGGACCC (35) |
| intron:Prkab1 | ACAGGAGACTCACTACACGGT (36) | AAATAGGGGGCAGGGACCAT (37) |
| exon:Gpc2 | GGAGATCATGTCAGACACCCC (38) | TGGAAGAAATGTGGTCAGCG (39) |
| intergenic:Gpa33- | TTTCATGCTCCTTGTTGTCGG (40) | TTGAATCCGGGCTCTATGGT (41) |
| Mael | | |
| intron:Wwox | CCTCCGCTGAGGTCTGAAGT (42) | GGCCCAACTGAACCCTAAGAT (43) |
| intergenic:CT573086.1- | GCTCCCTGCAGAAGGATCAC (44) | GACATCCACATGGCCTGTTC (45) |
| Hlcs | | |
| intergenic:U6atac- | TCAAGGTGGAGAAGGCATGG (46) | TCCTAAACCAGTATGAAAAGCTTCC (47) |
| 7SK | | |
| intergenic:Gm17566- | GGAAGCGGATTGCTGACATC (48) | CAGGGGAGGAACTAGAGGGAA (49) |
| AC124613.1 | | |
| intron:Hs6st3 | TGAGGCTCATAGGTTCACGTC (50) | ACAGAGACTCGAATCCCCCA (51) |
| intergenic:Irf2bp2- | GCCACACAGAAGGGGGTTAG (52) | TTAGGCCTGCATGGGAAAGG (53) |
| Tomm20 | | |
| intron:Sirpa | TTCCTGCTGAATGCCGTCAC (54) | TGTGATGCTTTAGGGAAAGATGC (55) |
| intergenic:Rmst-U7 | TCAGATGTGCAGGTCCAGAGA (56) | ATCCTTGTGCTTGCCCCTAT (57) |
| intron:Bmp6 | CACACTGCTCCTCTCCTGATT (58) | ACACAGCATGGAGTTCAAGCA (59) |
| intron:Kcnj10 | CAGCCACTTCACCTTCGAGC (60) | GATGGAAGACCCGAGGTGAATAA (61) |
| intron:Fras1 | GGCTATCTTTGGCTCGTCCA (62) | TCAAGAGGGTTCCAGTGGATT (63) |

*Numbers in parenthesis are SEQ ID Nos.

TABLE 6
Analysis of 20 off-target sites. Indel rates at the top 20 predicted off-target
sites (10 top off-target sites per sgRNA targeting exons 1 and 5 of the Sftpc
gene) as assessed by next-generation sequencing of lung DNA from 2 prenatal
Ad.Sftpc.GFP injected SFTPC^{173T/WT} fetuses at E19 (results separated by forward
slash) and a control uninjected SFTPC^{173T/WT} fetus harvested at E19.

| | sgRNA | | | Indels | |
|---|---|---|---|---|---|
| | Sftpc | | | Ad.Sftpc.GFP | |
| site | target | location | Sequence* | (n = 2) | Uninjected |
| OT1 | Exon 1 | intron:Ehd2 | TTGGAGCCCAGGCCCCAATA GGG (64) | 0.60%/0.70% | 0.53% |
| OT2 | Exon 1 | intron:Limk2 | TCAAACCCCAGGCCCAGATT AGG (65) | 0.08%/0.07% | 0.06% |
| OT3 | Exon 1 | intron:Actn4 | GCAGAGGCCAGGCCCAGATT AGG (66) | 0.28%/0.21% | 0.40% |
| OT4 | Exon 1 | Intron:Cars | TCTGATCCCAGGCCCAGTTA AGG (67) | 0.18%/0.15% | 0.16% |
| OT5 | Exon 1 | intergenic:Wnt9a- | ACAGAGCACAGGCCCAGAAA GGG (68) | 0.10%/0.11% | 0.10% |
| | | Prss38 | | | |
| OT6 | Exon 1 | intron:Hs6st3 | TAAGAGCCAAGGCCCCGACA GGG (69) | 0.15%/0.14% | 0.12% |
| OT7 | Exon 1 | intron:Prkab1 | TCAGAGCACAGGCTCAGAAA CGG (70) | 0.03%/0.02% | 0.03% |
| OT8 | Exon 1 | exon:Gpc2 | CCAGAGCCCAGGAACAGATA AGG (71) | 0.26%/0.23% | 0.20% |
| OT9 | Exon 1 | intergenic:Gpa33- | TCAGAGCCAAGGTCCCACTA TGG (72) | 0.16%/0.19% | 0.19% |
| | | Mael | | | |
| OT10 | Exon 1 | intron:Wwox | TCAAAGCCCAGGCCCAGCTT GGG (73) | 0.34%/0.35% | 0.39% |
| OT11 | Exon 5 | intergenic:CT573086.1- | ACAGGACCCACCTGGCCTGT TGG (74) | 0.23%/0.22% | 0.25% |
| | | Hlcs | | | |
| OT12 | Exon 5 | intergenic:U6atac- | ATAGGATACTCATGGCCCAT TGG (75) | 0.15%/0.13% | 0.10% |
| | | 7SK | | | |
| OT13 | Exon 5 | intergenic:Gm17566- | TTAAGATCTGCCTGGCCCGT GGG (76) | 0.09%/0.09% | 0.08% |
| | | AC124613.1 | | | |
| OT14 | Exon 5 | intron:Hs6st3 | ATAGGAGGCCCCAGGACCGT AGG (77) | 0.01%/0.01% | 0.01% |
| OT15 | Exon 5 | intergenic:Irf2bp2- | ATAGGACTGCCCTGGCCCTT TGG (78) | 0.16%/0.19% | 0.16% |
| | | Tomm20 | | | |
| OT16 | Exon 5 | intron:Sirpa | ATGGGATCCCCATGGACCGA GGG (79) | 0.14%/0.13% | 0.12% |
| OT17 | Exon 5 | intergenic:Rmst- | ATTGGATTCCCCAGGCCCGA AGG (80) | 0.18%/0.18% | 0.15% |
| | | U7 | | | |
| OT18 | Exon 5 | intron:Bmp6 | ATAGAAACCCCCAGGCCCCT CGG (81) | 0.24%/0.18% | 0.22% |
| OT19 | Exon 5 | intron:Kcnj10 | ATCGGATCCCCCTAACCCTT TGG (82) | 0.26%/0.24% | 0.22% |
| OT20 | Exon 5 | intron:Frasl | ATGGACTCCCCCTGGCCCTT AGG (83) | 0.18%/0.22% | 0.18% |

*Numbers in parenthesis are SEQ ID Nos.

### Prenatal Gene Editing in Sftpc^{I73T} Mutant Mice Improves Survival

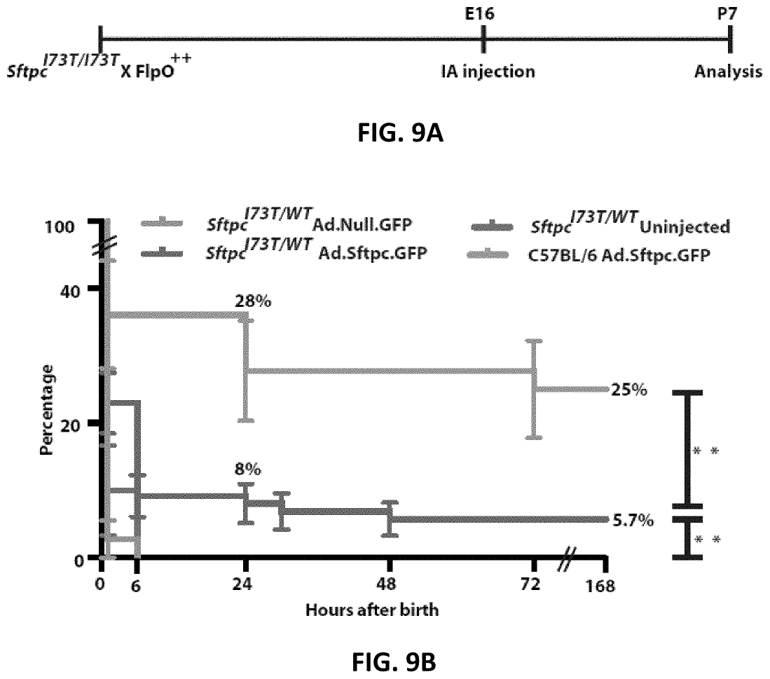

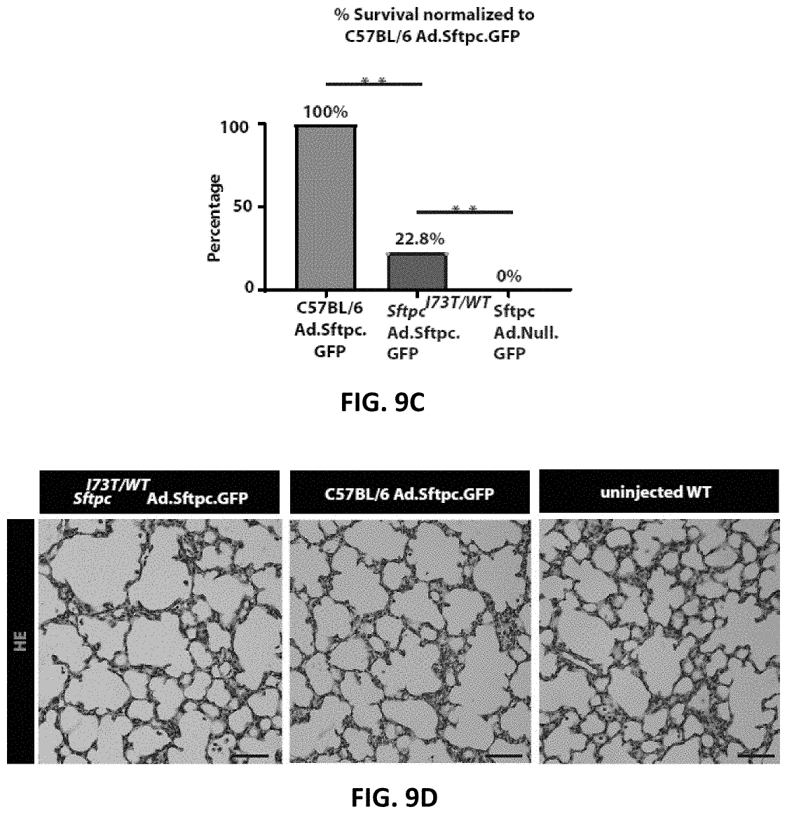

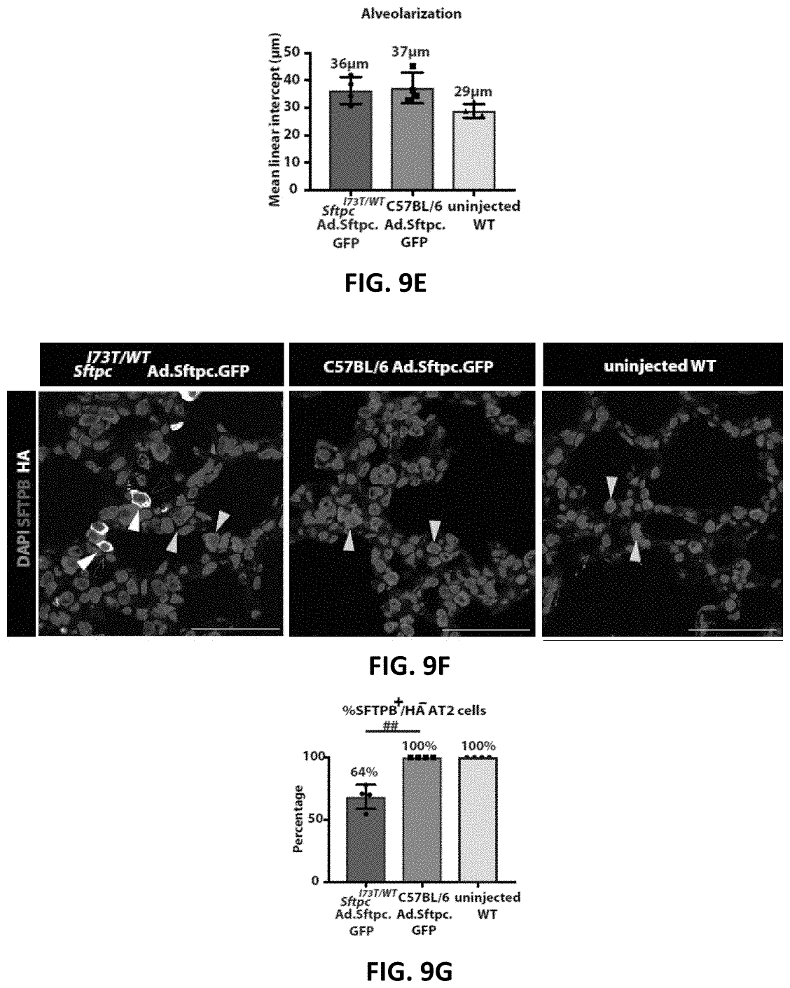

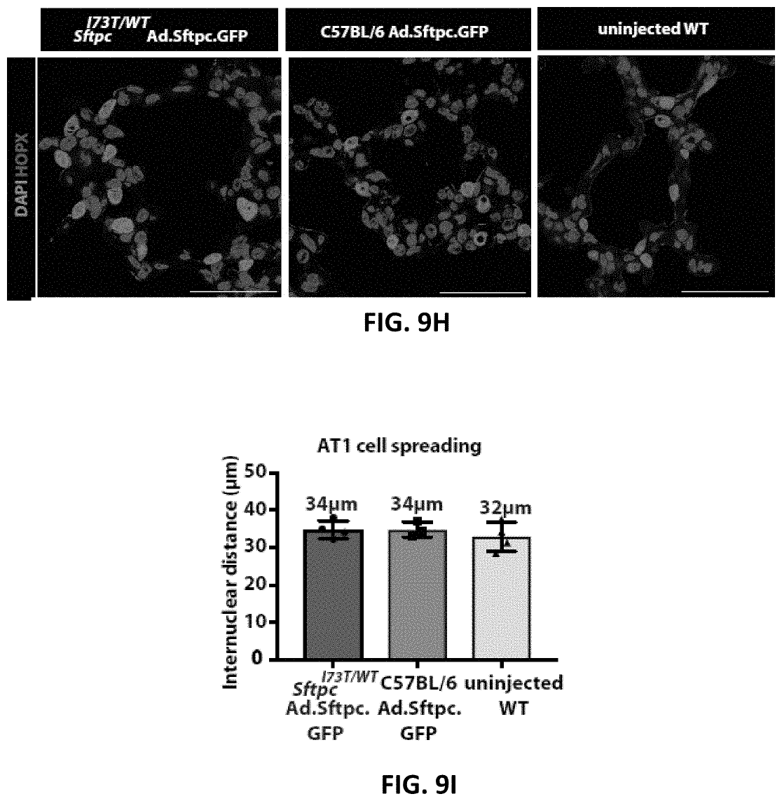

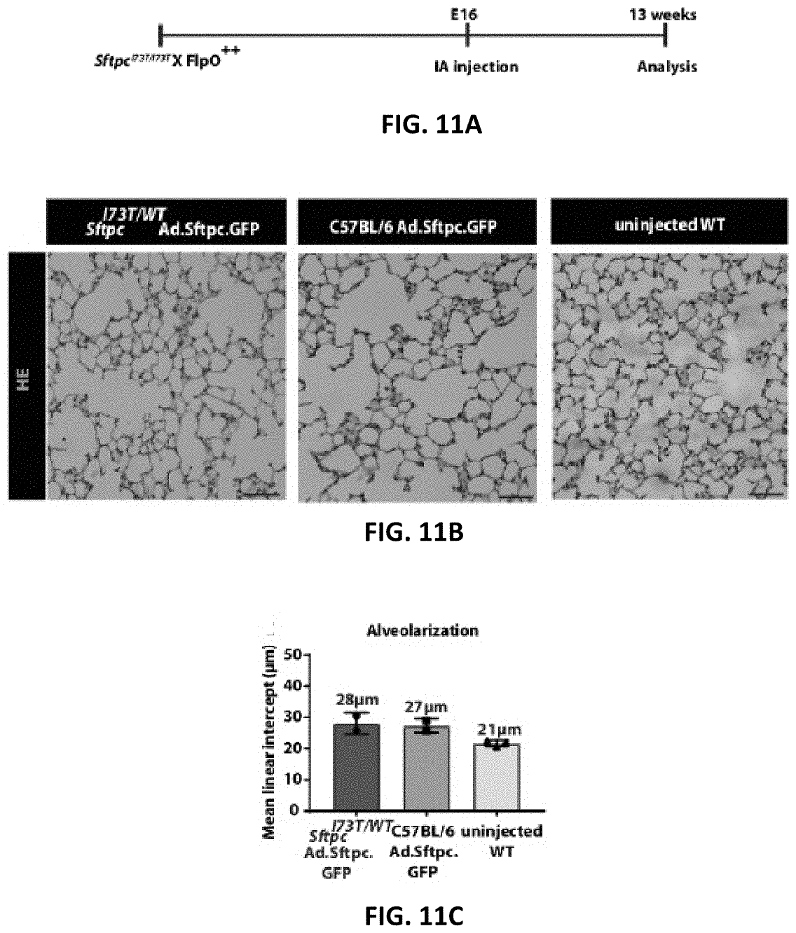

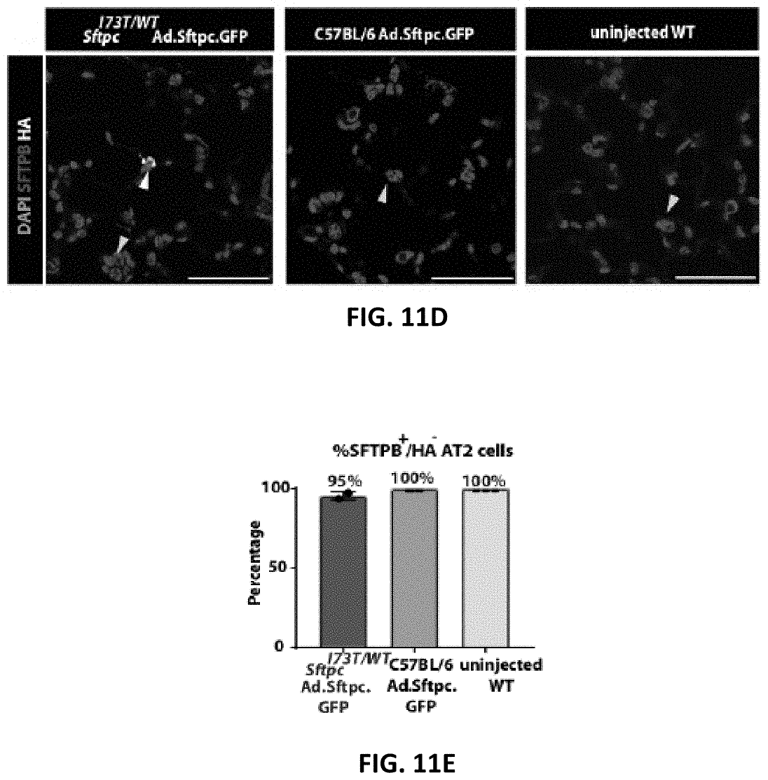

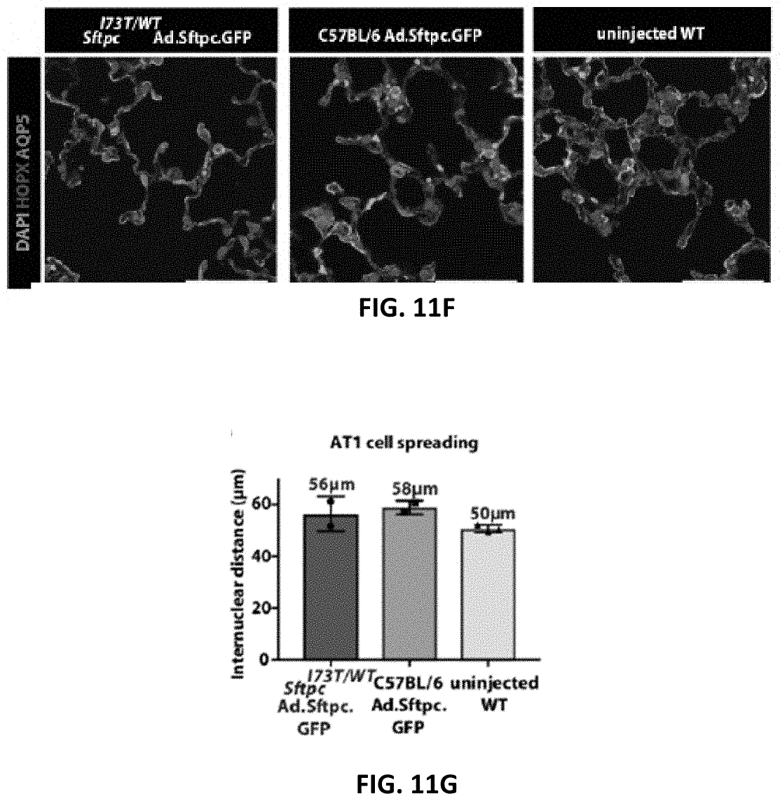

Because CRISPR-mediated excision of the Sftpc^{I73T} gene decreased the synthesis of the mutant SP-C^{I73T} pro-protein, improved AT2 and AT1 cell morphology and function, and improved lung maturation, we next tested if gene-edited mice exhibited improved survival. E16 Sftpc^{I73T/WT} fetuses were injected intra-amniotically with Ad.Null.GFP or Ad.Sftpc.GFP and assessed for survival up to one week of age (FIG. 9A). At baseline, this technique resulted in approximately 25% survival of C57BL/6 mice injected with Ad.Sftpc.GFP (n=9/36). Importantly, whereas none of the SftpC^{I73T/WT} fetuses injected with the control Ad.Null.GFP construct (n=0/36) survived beyond 6 hours after birth, a sizeable percentage of Sftpc^{I73T/WT} fetuses injected with Ad.Sftpc.GFP (n=7/87) survived beyond 24 hours (8%), including 5.7% (5/87) surviving to P7 (p=0.005), at which point they remained healthy as indicated by normal activity, respiratory effort, subjective growth, and the presence of a milk spot (visualized on PO through P2) indicative of feeding (FIG. 9B). Surviving animals were sacrificed at P7 to assess pulmonary histology. Using the C57BL/6 Ad.Sftpc.GFP-treated fetuses as a baseline, these data demonstrate a 22.8% improvement in survival of Sftpc^{I73T} mutant Ad.Sftpc.GFP-treated fetuses (FIG. 9C). In the surviving cohort, there was a marked improvement in lung alveolarization at P7, with comparable MLI between Sftpc^{I73T/WT} and C57BL/6 mice injected with Ad.Sftpc.GFP (FIGS. 9D and 9E). Sixty-eight percent of AT2 cells marked by SFTPB were HA-negative, which is similar to that demonstrated at E19 (FIGS. 9F and 9G), and AT1 cell spreading was comparable to that seen in C57BL/6 fetuses injected with Ad.Sftpc.GFP (FIGS. 9H and 9I). Furthermore, a limited number of rescued animals were analyzed at 13 weeks (FIG. 11A). MLI of Ad.Sftpc.GFP-injected SftpC^{I73T/WT} mice was comparable to Ad.Sftpc.GFP-injected C57BL/6 mice at this time point (FIG. 11B and FIG. 11C). Ninety-five percent of SFTPB AT2 cells were HA-negative in Ad.Sftpc.GFP-injected SftpC^{I73T/WT} mice, and the morphology of AT1 cells was comparable between Ad.Sftpc.GFP-injected Sftpc^{I73T/WT} and C57BL/6 mice (FIG. 11D-G). Our data demonstrate that fetal lung gene editing is feasible after intra-amniotic delivery of CRISPR-Cas9 and has the potential to attenuate embryonic toxic gain of function SP disease.

### Discussion

In this example, we demonstrate that CRISPR-Cas9 can be used to perform gene editing during tissue development through in utero intra-amniotic delivery to rescue a perinatal lethal monogenic lung disease. This approach targets the lung, with pulmonary epithelial cells including AT1, AT2, and secretory airway epithelial cells being preferentially edited. We show that in utero gene editing can ameliorate the phenotype of a congenital lung disease caused by the Sftpc^{I73T} mutation and improves survival of rescued mice. This study supports an important application of CRISPR-Cas9 to rescue viability at birth due to a lethal genetic mutation.

The design of the R26^{mTmG/+} model allowed for the tracking of cell type specificity, efficiency, and long-term persistence of gene editing. Our results demonstrate gene editing occurring predominantly in the lung and persisting up to 6 months of life, the last point of analysis. Using the intra-amniotic route of delivery, we were able to achieve approximately 20% editing in the lung epithelium, including the distally located AT1 and AT2 cells, at birth. Increased pulmonary epithelial cell editing compared to pulmonary endothelial and mesenchymal cell editing is likely due to direct contact between epithelial cells and the “inhaled” amniotic fluid as well as the location of adenovirus receptors on pulmonary epithelial cells that facilitates transduction (33, 42, 43).

In addition to pulmonary cell editing, we identified a few clusters of gene-edited cells in the proximal gastrointestinal tract after “swallowing” the amniotic fluid containing the viral vector, as previously demonstrated (33). Lower gastric compared to pulmonary cell editing might be due to the fairly rapid amniotic fluid inhalation, which was promoted by the administration of theophylline and maternal hypercarbia to enhance fetal breathing movements. Intra-amniotic injection might also be expected to target the skin. The lack of skin gene editing is likely explained by the skin barrier, formed initially by the periderm at E13 and completed by E17 after keratinization, which prevents viral vector transduction and thus epidermal editing after intra-amniotic delivery at E16 (31, 33). Lung-targeted gene editing is an advantage for genes that specifically cause lung disease, although they may be expressed in other organs. Thus, a targeted approach may minimize the exposure of other organs to potentially deleterious on- and off-target effects. Although lung-specific gene editing is beneficial for SP disease in the current study, alternative delivery approaches, including the intravenous route, may allow for efficient prenatal editing of other organs to address congenital genetic disorders that cause morbidity and mortality before or shortly after birth (27). Finally, although the use of theophylline and CO₂ to increase respiratory drive favored lung targeting in fetal mice via the intra-amniotic route, a more directed fetoscopic intra-tracheal approach could be performed in large animal models and in humans (44, 45).

Another advantage of in utero gene editing is the relatively uniform targeting of most of the major pulmonary epithelial cell types, including both proximal and distal lineages. In general, the inhalational route of drug delivery to postnatal lungs results in a differential distribution, with peripheral regions of the lungs receiving lower amounts compared to proximal and central regions (46). The efficiency of inhalational drug distribution is further impaired in the injured lung due to heterogeneity of lung disease, with some regions of the lung being overinflated and other regions collapsed. Thus, particularly for more complex lung disease, the more uniform distribution of vector delivery observed via an in utero intervention may provide an advantage in future therapies.

Given that many congenital lung diseases such as cystic fibrosis and inherited SP disease are generally caused by monogenic mutations, they should be ideal candidates for gene editing technologies. In mice, Sftpc expression is not required for survival and lung function at normal physiologic conditions (40), and thus a simple deletion of the mutant Sftpc^{I73T} gene was sufficient to improve mortality in our mouse model. Future therapeutic approaches in patients will likely require more targeted modifications in DNA. In humans, correction of the SFTPC^{I73T} mutation would be more desirable than excision of the mutated gene for treating the disease. However, our study demonstrates the feasibility of targeting the lung for gene editing before birth and presents evidence that even lethal mutations can be mitigated through prenatal gene editing techniques.

The use of Ad vectors is exemplified herein. Other delivery techniques, including AAV and/or lipid nanoparticles and smaller Cas9 genes can also be employed (18, 48, 49).

Although prenatal gene editing has the potential to take advantage of normal developmental properties to enhance editing efficiency and treat perinatal lethal diseases before birth, additional points must be considered that are not present for postnatal gene editing. Any prenatal intervention involves the possibility of affecting not only the fetus, but the mother who is an immunocompetent and often disease-free “bystander”. Thus, injection techniques and gene editing delivery vehicles can be optimized to avoid exposure to the mother. Given the potential maternal risk, initial disease targets should include those which cause major morbidity and/or mortality before or shortly after birth and for which no adequate treatments exist. Prenatal gene editing can involve mid to late gestation gene editing as detailed in the current study or early embryo gene editing. Early embryo gene editing can be performed ex vivo followed by implantation into the mother, thus avoiding maternal exposure to gene editing technology. In addition, early embryo gene editing may allow for more efficient correction of a larger number of cells in multiple organs, with the possibility of correcting germline cells. However, later gestation gene editing may allow editing to be more specific for a target organ or cell population, including avoiding germ cell editing, and would allow for the possibility of treating de novo mutations diagnosed later in pregnancy.

With the rapid pace at which CRISPR technology is advancing towards clinical translation, techniques that improve the efficiency and specificity of gene editing for targeting specific organs or tissues associated with specific diseases provide a new avenue for treatment of such disorders. Our studies demonstrate the feasibility of prenatal gene editing with high specificity for the lung represent a promising approach to address the unmet need for therapeutic approaches to congenital lung diseases that are fatal at birth.

### REFERENCES

1. A. Hamvas, F. S. Cole, L. M. Nogee, Genetic disorders of surfactant proteins. Neonatology 91, 311-317 (2007).
2. J. A. Whitsett, S. E. Wert, T. E. Weaver, Alveolar surfactant homeostasis and the pathogenesis of pulmonary disease. Annual review of medicine 61, 105-119 (2010).
3. S. M. Rowe, S. Miller, E. J. Sorscher, Cystic fibrosis. The New England journal of medicine 352, 1992-2001 (2005).
4. R. G. Crystal, Alpha 1-antitrypsin deficiency, emphysema, and liver disease. Genetic basis and strategies for therapy. The Journal of clinical investigation 85, 1343-1352 (1990).
5. K. A. Spoonhower, P. B. Davis, Epidemiology of Cystic Fibrosis. Clinics in chest medicine 37, 1-8 (2016).
6. H. A. Tanash, P. M. Nilsson, J. A. Nilsson, E. Piitulainen, Clinical course and prognosis of never-smokers with severe alpha-1-antitrypsin deficiency (PiZZ). Thorax 63, 1091-1095 (2008).
7. H. S. Cameron, M. Somaschini, A common mutation in the surfactant protein C gene associated with lung disease. The Journal of pediatrics 146, 370-375 (2005).
8. L. M. Nogee, Interstitial lung disease in newborns. Seminars in fetal & neonatal medicine 22, 227-233 (2017).
9. W. B. Eldridge, Q. Zhang, A. Faro, S. C. Sweet, P. Eghtesady, A. Hamvas, F. S. Cole, J. A. Wambach, Outcomes of Lung Transplantation for Infants and Children with Genetic Disorders of Surfactant Metabolism. The Journal of pediatrics 184, 157-164 (2017).
10. S. Kirkby, D. Hayes, Jr., Pediatric lung transplantation: indications and outcomes. Journal of thoracic disease 6, 1024-1031 (2014).
11. J. A. Doudna, E. Charpentier, Genome editing. The new frontier of genome engineering with CRISPR-Cas9. Science 346, 1258096 (2014).
12. F. A. Ran, P. D. Hsu, J. Wright, V. Agarwala, D. A. Scott, F. Zhang, Genome engineering using the CRISPR-Cas9 system. Nature protocols 8, 2281-2308 (2013).
13. P. Mali, K. M. Esvelt, G. M. Church, Cas9 as a versatile tool for engineering biology. Nature methods 10, 957-963 (2013).
14. H. Ma, N. Marti-Gutierrez, S. W. Park, J. wu, Y. Lee, K. Suzuki, A. Koski, D. Ji, T. Hayama, R. Ahmed, H. Darby, C. Van Dyken, Y. Li, E. Kang, A. R. Park, D. Kim, S. T. kim, J. Gong, Y. Gu, X. Xu, D. Bataglia, S. A. Krieg, D. M. Lee, D. H. Wu, D. P. Wolf, S. B. Heitner, J. C. I. Belmonte, P. Amato, J. S. Kim, S. Kaul, S. Mitalipov, Correction of a pathogenic gene mutation in human embryos. Nature 548, 413-419 (2017).
15. S. Q. Tsai, J. K. Joung, Defining and improving the genome-wide specificities of CRISPR-Cas9 nucleases. Nature reviews. Genetics 17, 300-312 (2016).
16. C. E. Nelson, C. H. Hakim, D. G. Ousterout, P. I. Thakore, E. A. Moreb, R. M. Castellanos Rivera, S. Madhavan, X. Pan, F. A. Ran, W. X. Yan, A. Asokan, F. Zhang, D. Duad, C. A. Gersbach, In vivo genome editing improves muscle function in a mouse model of Duchenne muscular dystrophy. Science 351, 403-407 (2016).
17. M. Tabebordbar, K. Zhu, J. K. Cheng, W. L. Chew, J. J. Widrick, W. X. Yan, C. Masener, E. Y. Wu, R. Xiao, F. A. Ran, L. Cong, F. Zhang, L. H. Vandenberghe, G. M. Church, A. J. Wagers, In vivo gene editing in dystrophic mouse muscle and muscle stem cells. Science 351, 407-411 (2016).
18. Y. Yang, L. Wang, P. Bell, D. McMenamin, Z. He, J. White, H. Yu, C. Xu, H. Morizono, K. Musunuru, M. L. Batshaw, J. M. Wilson, A dual AAV system enables the Cas9-mediated correction of a metabolic liver disease in newborn mice. Nat Biotechnol 34, 334-338 (2016).
19. C. Q. Song, D. Wang, T. jiang, K. O'Connor, Q. Tang, L. Cai, X. Li, Z. Weng, H. Yin, G. Gao, C. Mueller, T. R. Flotte, W. Xue, In vivo genome editing partially restores alpha-a antitrypsin in a murine model of AAT deficiency, Hum Gene Ther 29, 853-860 (2018).
20. Y. Wu, D. Liang, Y. Wang, M. Bai, W. Tang, S. Bao, Z. Yan, D. Li, J. Li, Correction of a genetic disease in mouse via use of CRISPR-Cas9. Cell Stem Cell 13, 659-662 (2013).
21. M. El Refaey, L. Xu, Y. Gao, B. D. Canan, T. M. A. Adesanya, S. C. Warner, K. Akagi, D. E. Symer, P. J. Mohler, J. Ma, P. M. L. Janssen, R. Han. In Vivo Genome Editing Restores Dystrophin Expression and Cardiac Function in Dystrophic Mice. Circ Res 121, 923-929 (2017).
22. N. M. E. Fogarty, A. McCarthy, K. E. Snijders, B. E. Powell, N. Kubikova, P. Blakeley, R. Lea, K. Elder, S. E. Wamaitha, D. Kim, V. Maciulyte, J. Kleinjung, J. S. Kim, D. Wells, L. Vallier, A. Bertero, J. M. A. Turner, K. K. Niakan, Genome editing reveals a role for OCT4 in human embryogenesis. Nature 550, 67-73 (2017).
23. C. Long, J. R. McAnally, J. M. Shelton, A. A. Mireault, R. Bassel-Duby, E. N. Olson, Prevention of muscular dystrophy in mice by CRISPR/Cas9-mediated editing of germline DNA. Science (New York, N.Y.) 345, 1184-1188 (2014).
24. J. Wu, M Vilarino, K. Suzuki, D. Okamura, Y. S. Bogliotti, I. Park, J. Rowe, B. McNabb, P. J. Ross, J. C. I. Belmonte, CRISPR-Cas9 mediated one-step disabling of pancreatogenesis in pigs. Sci Rep 7, 10487 (2017).
25. M. S. Carlon, D. Vidovic, J. Dooley, M. M. da Cunha, M. Maris, Y. Lampi, J. Toelen, C.
Van den Haute, V. Baekelandt, J. Deprest, E. Verbeken, A. Liston, R. Gijsbers, Z. Debyser, Immunological ignorance allows long-term gene expression after perinatal recombinant adeno-associated virus-mediated gene transfer to murine airways. Hum Gene Ther 25, 517-528 (2014).
26. M. G. Davey, J. S. Riley, A. Andrews, A. Tyminski, M. Limberis, J. E. Pogoriler, E. Patridge, A. Olive, H. L. Hedrick, A. W. Flake, W. H. Peranteau, Induction of Immune Tolerance to Foreign Protein via Adeno-Associated Viral Vector Gene Transfer in Mid-Gestation Fetal Sheep. PLoS One 12, e0171132 (2017).
27. A. C. Rossidis, J. D. Stratigis, A. C. Chadwick, H. A. Hartman, N. J. Ahn, H. Li, K. Singh, B. E. Coons, L. Li, W. Lv, P. W. Zoltick, D. Alapati, W. Zacharias, R. Jain, E. E. Morrisey, K. Musunuru, W. H. Peranteau, In utero CRISPR-mediated therapeutic editing of metabolic genes. Nat Med 24, 1513-1518 (2018).
28. D. E. Sabatino, T. C. Mackenzie, W. Peranteau, S. Edmonson, C. Campagnoli, Y. L. Liu,
A. W. Flake, K. A. High, Persistent expression of hF.IX After tolerance induction by in utero or neonatal administration of AAV-1-F.IX in hemophilia B mice. Molecular therapy: the journal of the American Society of Gene Therapy 15, 1677-1685 (2007).
29. G. A. Duncan, J. Jung, J. Hanes, J. S. Suk, The Mucus Barrier to Inhaled Gene Therapy. Molecular therapy: the journal of the American Society of Gene Therapy 24, 2043-2053 (2016).
30. P. L. Sinn, E. R. Burnight, P. B. McCray, Jr., Progress and prospects: prospects of repeated pulmonary administration of viral vectors. Gene therapy 16, 1059-1065 (2009).
31. M. Endo, T. Henriques-Coelho, P. W. Zoltick, D. H. Stitelman, W. H. Peranteau, A. Radu, A. W. Flake, The developmental stage determines the distribution and duration of gene expression after early intra-amniotic gene transfer using lentiviral vectors. Gene Ther 17, 61-71 (2010).
32. L. Joyeux, E. Danzer, M. P. Limberis, P. W. Zoltick, A. Radu, A. W. Flake, M. G. Davey, In utero lung gene transfer using adeno-associated viral and lentiviral vectors in mice. Hum Gene Ther Methods 25, 197-205 (2014).
33. S. M. Buckley, S. N. Waddington, S. Jezzard, L. Lawrence, H. Schneider, M. V. Holder, M. Themis, C. Coutelle, Factors influencing adenovirus-mediated airway transduction in fetal mice. Molecular therapy: the journal of the American Society of Gene Therapy 12, 484-492 (2005).
34. M. D. Muzumdar, B. Tasic, K. Miyamichi, L. Li, L. Luo, A global double-fluorescent Cre reporter mouse. Genesis 45, 593-605 (2007).
35. S. I. Nureki, Y. Tomer, A. Venosa, L. Katzen, S. J. Russo, S. Jamil, M. Barrett, V. Nguyen, M. Kopp, S. Mulugeta, M. F. Beers, Expression of mutant Sftpc in murine alveolar epithelia drives spontaneous lung fibrosis. The Journal of clinical investigation 128, 4008-4024 (2018).
36. M. Kosicki, K. Tomberg, A. Bradley, Repair of double-strand breaks induced by CRISPR-Cas9 leads to large deletions and complex rearrangements. Nat Biotechnol 36, 765-771 (2018).
37. F. Brasch, M. Griese, M. Tredano, G. Johnen, M. Ochs, C. Rieger, S. Mulugeta, K. M. Muller, M. Bahuau, M. F. Beers, Interstitial lung disease in a baby with a de novo mutation in the SFTPC gene. Eur Respir J 24, 30-39 (2004).
38. M. F. Beers, A. Hawkins, J. A. Maguire, A. Kotorashvili, M. Zhao, J. L. Newitt, W. Ding, S. Russo, S. Guttentag, S. Gonzales, S. Mulugeta, A nonaggregating surfactant protein C mutant is misdirected to early endosomes and disrupts phospholipid recycling. Traffic 12, 1196-1210 (2011).
39. A. Hawkins, S. Guttentag, R. Deterding, W. K. Funkhouser, J. L. Goralski, S. Chatterjee, S. Mulugeta, M. F. Beers, A non-BRICHOS SFTPC mutant (SP-CI73T) linked to interstitial lung disease promotes a late block in macroautophagy disrupting cellular proteostasis and mitophagy. Am J Physiol Lung Cell Mol Physiol 308, L33-47 (2015).
40. S. W. Glasser, M. S. Burhans, T. R. Korfhagen, C. L. Na, P. D. Sly, G. F. Ross, M. Ikegami, J. A. Whitsett, Altered stability of pulmonary surfactant in SP-C-deficient mice. Proceedings of the National Academy of Sciences of the United States of America 98, 6366-6371 (2001).
41. M. Haeussler, K. Schonig, H. Eckert, A. Eschstruth, J. Mianne, J. B. Renaud, S.
Schneider-Maunoury, A. Shkumatava, L. Teboul, J. Kent, J. S. Joly, J. P. Concordet, Evaluation of off-target and on-target scoring algorithms and integration into the guide RNA selection tool CRISPOR. Genome Biol 17, 148 (2016).
42. N. Arnberg, Adenovirus receptors: implications for targeting of viral vectors. Trends Pharmacol Sci 33, 442-448 (2012).
43. A. L. Cooney, B. K. Singh, L. M. Loza, I. M. Thornell, C. E. Hippee, L. S. Powers, L. S. Ostedgaard, D. K. Meyerholz, C. Wohlford-Lenane, D. A. Stoltz, P. Jr. B McCray, P. L. Sinn, Widespread airway distribution and short-term phenotypic correction of cystic fibrosis pigs following aerosol delivery of piggyBac/adenovirus. Nucleic Acids Res 46, 9591-9600 (2018).
44. A. L. David, D. M. Peebles, L. Gregory, M. Themis, T. Cook, C. Coutelle, C. H. Rodeck, Percutaneous ultrasound-guided injection of the trachea in fetal sheep: a novel technique to target the fetal airways. Fetal Diagn Ther 18, 385-390 (2003).
45. M. R. Harrison, R. L. Keller, S. B. Hawgood, J. A. Kitterman, P. L. Sandberg, D. L. Farmer, H. Lee, R. A. Filly, J. A. Farrell, C. T. Albanese, A randomized trial of fetal endoscopic tracheal occlusion for severe fetal congenital diaphragmatic hernia. The New England journal of medicine 349, 1916-1924 (2003).
46. N. R. Labiris, M. B. Dolovich, Pulmonary drug delivery. Part I: physiological factors affecting therapeutic effectiveness of aerosolized medications. Br J Clin Pharmacol 56, 588-599 (2003).
47. Y. S. Ahi, D. S. Bangari, S. K. Mittal, Adenoviral vector immunity: its implications and circumvention strategies. Curr Gene Ther 11, 307-320 (2011).
48. E. Kim, T. Koo, S. W. Park, D. Kim, K. Kim, H. Y. Cho, D. W. Song, K. J. Lee, MH. Jung,
S. Kim, J. H. Kim, J. S. Kim, In vivo genome editing with a small Cas9 orthologue derived from Campylobacter jejuni. Nat Commun 8, 14500 (2017).
49. H. Yin, C. Q. Song, S. Suresh, Q. Wu, S. Walsh, L. H. Rhym, E. Mintzer, M. F. Bolukbasi, L. J. Zhu, K Kauffman, H. Mou, A. Oberholzer, J. Ding, S. Y. Kwan, R. L. Bogorad, T. Zatsepin, V. Koteliansky, S. A. Wolfe, W. Xue, R. Langer, D. G. Anderson, Structure-guided chemical modification of guide RNA enables potent non-viral in vivo genome editing. Nat Biotechnol 35, 1179-1187 (2017).
50. L. Cong, F. A. Ran, D. Cox, S. Lin, R. Barretto, N. Habib, P. D. Hsu, X. wu, W. Jiang, L. A. Marraffini, F. Zhang, Multiplex genome engineering using CRISPR/Cas systems. Science 339, 819-823 (2013).
51. J. A. Zepp et al., Distinct Mesenchymal Lineages and Niches Promote Epithelial Self-Renewal and Myofibrogenesis in the Lung. Cell 170, 1134-1148 (2017).
52. Y. Wang, D. B. Frank, M. P. Morley, S. Zhou, X. Wang, M. M. Lu, M. A. Lazar, E. E. Morrisey, HDAC3-Dependent Epigenetic Pathway Controls Lung Alveolar Epithelial Cell Remodeling and Spreading via miR-17-92 and TGF-beta Signaling Regulation. Dev Cell 36, 303-315 (2016).

### Example II

### Gene Editing in the Lung of the Sheep Fetus

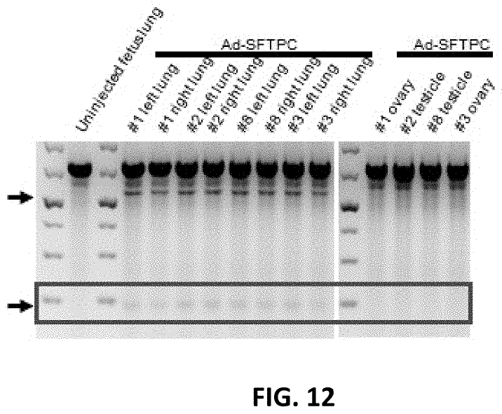

The results presented in the previous example, indicate that gene editing could have a significant impact on monogenic lung disease in larger animal subjects and humans. In this example, we describe compositions and methods for targeting the developing lung in a large animal model (e.g., fetal sheep) which is relevant to human treatment. Fetal sheep were injected via the intratracheal route with an adenoviral vector containing SpCas9 and a guide RNA targeting the ovine SPC gene using an open technique. This approach entails performing a maternal laparotomy and opening the uterus. The fetal trachea was then identified and injected with a biologically compatible solution comprising the viral vector, in this example, an adenovirus. Fetal organs were harvested at 7-10 days post injection and DNA was assessed by surveyor and next generation sequencing for editing at the target sites. This demonstrated editing efficiencies of approximately 5% of all pulmonary cells (FIG. 12).

In another approach, other vectors may be used to carry the gene therapy or gene editing material. These vectors include, without limitation, adeno-associated viruses, retroviral constructs, and nanoparticle technology. Additionally, a minimally invasive approach of fetoscopic access to the fetus may be used throughout gestation with subsequent cannulation of the trachea and injection of the therapy after which temporary closure of the trachea would be performed or, alternatively, no closure of the trachea would be performed.

### Example III

### Gene Editing in the Lung of the Human Fetus

The results presented in the previous examples, indicate that gene editing could have a significant impact on monogenic lung disease in human subjects. Potential target diseases include without limitation, Cystic fibrosis, Surfactant protein deficiencies including for example, surfactant protein C deficiency, surfactant protein B deficiency, and ABCA3 deficiency, and Alveolar capillary dysplasia, and Alpha-1-antitrypsin disease.

Hereditary surfactant protein B (SP-B) deficiency is an autosomal recessive disorder that causes fatal respiratory failure in the neonatal period. Full-term infants born with SF-B deficiency have respiratory failure and the disease is fatal by 3-6 months of age. Currently, the only treatment for SP-B deficiency is a lung transplant. Carriers of the disease are asymptomatic. Although, more than 40 distinct mutations in the SP-B gene have been identified, two thirds of the mutant disease-causing alleles result from the 121ins2 mutation (Ref SNP: rs35328240) in exon 4. The mutation consists of a net 2-base pair insertion in exon 4 of the SFTPB gene (375C-GAA change) resulting in a frameshift and premature termination of the protein.

Using SP-B deficiency as an example, it is clear that clinical application of in utero gene editing entailing the use of targeted CRISPR-Cas9 mediated homology directed repair to correct the common 121ins2 mutation in the SFTPB gene (which causes lethal respiratory failure shortly after birth), is feasible. Parents can be initially screened for the disease-causing mutations in order to identify carriers of the mutated gene. In pregnancies from carriers identified as having of the mutant allele, CVS or other diagnostic modalities can be offered to identify the presence of a homozygous mutation in the fetus. Once an affected fetus is identified, in utero gene editing can be offered to the parents.

In one approach, during mid-gestation (e.g., between 20 and 28 weeks of pregnancy, preferably at 20 weeks, 22 weeks, 24 weeks, or 26 weeks of gestation), a fetoscope can be used to introduce into the fetal airway a catheter comprising an insufflated balloon with an injection port distal to the balloon. The balloon can be deployed to block the airway to prevent the escape of the gene editing system which is injected as a bolus immediately after balloon deployment. After approximately 1 week following delivery of the system, a second procedure can be performed to remove the balloon, thereby eliminating obstruction of the airway to preclude any issues at birth and to allow for continued development of the lungs without tracheal occlusion. This is significant as tracheal occlusion is known to affect normal development of the lung and is associated with abnormal surfactant production. Follow up clinical studies can be performed to ensure that the mutated gene has been corrected and normal phenotypes restored.

In another approach, fetuses harboring the delta 508 mutation in CF or the I73T mutation associated with surfactant protein C deficiency, or the myriad of other, less prevalent mutations present in CF and the surfactant protein deficiencies can be treated using gene editing systems comprising reagents suitable for correcting these genetic mutations. In yet other approaches, post-natal infants can be treated employing a vector system described herein. In on aspect of this method, the vector is delivered in aerosolized form, or via an inhaler/nebulizer. Another method entails direct injection of vector containing biologically compatible liquids directly into the airways via a bronchoscopy

Several different approaches are available to the person of skill in this art area for delivering the genetic editing systems described herein. These include without limitation:

1. Viral vectors such as adenovirus, lentivirus, AAV virus (including the multiple different serotypes), lentivirus
2. Non-viral delivery techniques, e.g., loaded exosomes, nanofiber and nanoparticle delivery approaches described above.

Human target genes and GenBank Reference numbers of relevant gene sequences include for example, ABCA3—NG_011790.1; SFTPC—NG_016968.1; SFTPB—NG_016967.1; CFTR—NG_016465.4. CFTR and SERPINA1—NP_000286.3.

Guide strands useful in the methods described for editing the Surfactant protein C can be selected from:

I73T-SFTPC-gRNA1
(SEQ ID NO. 84)
GTGCTCATCTCCAGAACCTGGGG;
I73T-SFTPC-gRNA2
(SEQ ID NO. 85)
AGTGCTCATCTCCAGAACCTGGG;
I73T-SFTPC-gRNA3
(SEQ ID NO. 86)
CAGTGCTCATCTCCAGAACCTGG;
I73T-SFTPC-gRNA4
(SEQ ID NO. 87)
CAGGTTCTGGAGATGAGCACTGG;
I73T-SFTPC-gRNA5
(SEQ ID NO. 88)
AGGTTCTGGAGATGAGCACTGGG;
I73T-SFTPC-gRNA6
(SEQ ID NO. 89)
GGTTCTGGAGATGAGCACTGGGG;
I73T-SFTPC-gRNA7
(SEQ ID NO. 90)
GGAGATGAGCACTGGGGCGCCGG;
I73T-SFTPC-gRNA8
(SEQ ID NO. 91)
GAGATGAGCACTGGGGCGCCGG;
I73T-SFTPC-gRNA9
(SEQ ID NO. 92)
AGATGAGCACTGGGGCGCCGGA;
I73T-SFTPC-gRNA10
(SEQ ID NO. 93)
GAGATGAGCACTGGGGCGCCGGA;
I73T-SFTPC-gRNA11
(SEQ ID NO. 94)
ATGAGCACTGGGGCGCCGGAAG;
I73T-SFTPC-gRNA12
(SEQ ID NO. 95)
CACTGGGGCGCCGGAAGCCCAG;
I73T-SFTPC-gRNA13
(SEQ ID NO. 96)
TCTGGAGATGAGCACTGGGGCG;
I73T-SFTPC-gRNA14
(SEQ ID NO. 97)
GTTCTGGAGATGAGCACTGGGG;
and
I73T-SFTPC-gRNA15
(SEQ ID NO. 98)
CAGGTTCTGGAGATGAGCACTG.

Guide strands useful in the methods described for editing the CFTR can be selected from

CFTR-Del508-gRNA1
(SEQ ID NO. 99)
ACCATTAAAGAAAATATCATTGG;
CFTR-Del508-gRNA2
(SEQ ID NO. 100)
ACCAATGATATTTTCTTTAATGG;
CFTR-Del508-gRNA3
(SEQ ID NO. 101)
TCTGTATCTATATTCATCATAGG;
CFTR-Del508-gRNA4
(SEQ ID NO. 102)
AATGGTGCCAGGCATAATCCAGG;
and
CFTR-Del508-gRNA5
(SEQ ID NO. 103)
AGTTTCTTACCTCTTCTAGTTGG.

In preferred embodiments, non-integrating viral vectors or nanoparticles are employed to deliver the CRISPR-Cas9 system, eliminating the need for removal of the system as expression of the system should be transient.

As an alternative approach, base editing, a form of gene editing, can be used to correct disease causing mutations in congenital genetic lung diseases. For surfactant protein C deficiency, the most common disease-causing mutation is a T→C base change resulting in a gain-of-function mutation. Using cytosine deaminase base editors (CBE), which can change a C→T, we have identified guide RNAs and CBEs that can correct the mutation in a mouse model of Surfactant protein C deficiency that has the most common human mutation. The human SPC gene sequence has been screened and multiple gRNAs have been identified that have the potential to change the disease causing C→T mutation with CBE (Table 7).

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

What is claimed is:

1. A CRISPR-Cas system-mediated genome editing method comprising introducing into a eukaryotic cell containing and expressing a DNA molecule having a target sequence and encoding at least one mutated gene product in the lung, an engineered, non-naturally occurring CRISPR-Cas system comprising one or more vectors comprising:
a) a first regulatory element operable in a eukaryotic cell operably linked to at least one nucleotide sequence encoding a CRISPR-Cas system guide RNA that hybridizes with the target sequence, and
b) a second regulatory element operable in a eukaryotic cell operably linked to a nucleotide sequence encoding a Cas9 protein or variant thereof, wherein components (a) and (b) are located on same or different vectors of the system or are affixed molecules which effect nucleic acid delivery into mammalian cells, whereby expression of the at least one gene product is altered through the CRISPR-Cas system acting via the DNA molecule comprising the guide RNA directing sequence-specific binding of the CRISPR-Cas system, causing genome editing to remove one or more undesired mutations; and, wherein the Cas9 protein and the guide RNA do not naturally occur together, and said guide strand targets a gene in fetal or post-natal lung selected from the group consisting mutated SFTPB, SFTPC, ABCA3, SERPINA1, and CFTR.

2. The method of claim 1, wherein said target sequence is present in:
i) mutated SFTPB or SFTPC;
ii) mutated CFTR;
iii) mutated ABCA3; or
iv) SERPINA1.

3.-5. (canceled)

6. The method of claim 1, wherein the CRISPR-Cas system further comprises one or more nuclear localization sequence(s).

7. The method of claim 6, wherein the CRISPR-Cas system comprises a tracr sequence.

8. The method of claim 7, wherein the Cas9 protein is codon optimized for expression in the eukaryotic lung cell.

9. The method of claim 1, wherein the eukaryotic cell is a mammalian or human cell.

10. The method of claim 1 for correcting a mutation causing monogenic lung disease in a subject in need thereof, the method comprising delivering said CRISPR-Cas system-mediated genome editing system to the lungs of said subject, thereby editing a mutated gene in the lungs and ameliorating symptoms of said monogenic lung disease.

11. The method of claim 10, wherein said subject is selected from a fetal, post-natal, pediatric or adult subject.

12. A CRISPR/Cas nuclease comprising a single guide RNA that binds to a target site in a mutated gene causing monogenic lung disease, wherein the nuclease cleaves and inactivates the mutated gene, wherein said gene is SFTPC or CFTR.

13. The CRISPR/Cas nuclease of claim 12, wherein said gene is SFTPC and said guide RNA is selected from SEQ ID NOS: 84-98.

14. The CRISPR/Cas nuclease of claim 12, wherein said gene is CFTR and said guide RNA is selected from SEQ ID NOS: 99-103.

15. A mammalian cell comprising the nuclease of claim 12.

16. A method of inactivating an endogenous gene causing monogenic disease in a lung cell, the method comprising the steps of: administering to the cell a CRISPR/Cas nuclease according to claim 13, wherein the nuclease cleaves and inactivates a gene causing lethal monogenic lung disease.

17. A guide strand for use in the method of claim 1, where the gene is Surfactant protein C and said sgRNAs are selected from,
I73T-SFTPC-gRNA1
GTGCTCATCTCCAGAACCTGGGG;
I73T-SFTPC-gRNA2
AGTGCTCATCTCCAGAACCTGGG;
I73T-SFTPC-gRNA3
CAGTGCTCATCTCCAGAACCTGG;
I73T-SFTPC-gRNA4
CAGGTTCTGGAGATGAGCACTGG;
I73T-SFTPC-gRNA5
AGGTTCTGGAGATGAGCACTGGG;
I73T-SFTPC-gRNA6
GGTTCTGGAGATGAGCACTGGGG;
I73T-SFTPC-gRNA7
GGAGATGAGCACTGGGGCGCCGG;
I73T-SFTPC-gRNA8
GAGATGAGCACTGGGGCGCCGG;
I73T-SFTPC-gRNA9
AGATGAGCACTGGGGCGCCGGA;
I73T-SFTPC-gRNA10
GAGATGAGCACTGGGGCGCCGGA;
I73T-SFTPC-gRNA11
ATGAGCACTGGGGCGCCGGAAG;
I73T-SFTPC-gRNA12
CACTGGGGCGCCGGAAGCCCAG;
I73T-SFTPC-gRNA13
TCTGGAGATGAGCACTGGGGCG;
I73T-SFTPC-gRNA14
GTTCTGGAGATGAGCACTGGGG;
and
I73T-SFTPC-gRNA15
CAGGTTCTGGAGATGAGCACTG.

18. A guide strand for use in the method of claim 1, where the gene is CFTR said sgRNAs are selected from
CFTR-Del508-gRNA1
ACCATTAAAGAAAATATCATTGG;
CFTR-Del508-gRNA2
ACCAATGATATTTTCTTTAATGG;
CFTR-Del508-gRNA3
TCTGTATCTATATTCATCATAGG;
CFTR-Del508-gRNA4
AATGGTGCCAGGCATAATCCAGG;
and
CFTR-Del508-gRNA5
AGTTTCTTACCTCTTCTAGTTGG.

19. A kit for practicing the method of claim 1, comprising A CRISPR/Cas nuclease comprising a single guide RNA that binds to a target site in a mutated gene causing monogenic lung disease, and means for delivering said nuclease into a lung cell.

20. The kit of claim 19, wherein said nuclease is formulated for aerosolized delivery to the lung.

21. The kit of claim 19, wherein said nuclease is formulated in a biocompatible liquid vehicle for delivery to the lung via bronchoscopy.

22. A method of inactivating an endogenous gene causing monogenic disease in a lung cell, the method comprising the steps of: administering to the cell a CRISPR/Cas nuclease according to claim 14, wherein the nuclease cleaves and inactivates a gene causing lethal monogenic lung disease.
